(19) United States
(12) Patent Application Publication
(10) Pub. No.: US 2019/0125988 A1
(43) Pub. Date: May 2, 2019

(54) METHOD AND DEVICE FOR VAPORIZING PHYTO MATERIAL

(71) Applicant: Michael Alexander Trzecieski, Toronto (CA)

(72) Inventor: Michael Alexander Trzecieski, Toronto (CA)

(21) Appl. No.: 16/172,193

(22) Filed: Oct. 26, 2018

Related U.S. Application Data
(60) Provisional application No. 62/577,758, filed on Oct. 27, 2017.

(51) Int. Cl. A61M 11/04; A61M 15/06; A61M 15/00

(52) U.S. Cl. CPC A61M 11/042; A61M 15/06; A61M 2205/3606; A61M 2205/3368; A61M 15/0025

(57) ABSTRACT

A device and method for vaporizing phyto material. The vaporization device has a body that includes a heating chamber and a heating element usable to selectively heat different regions within the heating chamber. The heating chamber has perforated walls that define a chamber cavity. A lid is movably mounted to the body. The lid is moveable between open and closed positions. The lid has a cooling element positioned therein and an inhalation aperture that is fluidly coupled to the air cooler. In the open position, phyto material is loadable into the chamber cavity. In the closed position, the heating element is energizable to heat and vaporize the phyto material. In some embodiments, the chamber cavity is divided into a plurality of segments along the length of the heating chamber and the heating element is movable to heat and vaporize the phyto material in a specific segment.

### CROSS-REFERENCE TO RELATED APPLICATIONS

This application claims the benefit of U.S. Provisional Application No. 62/577,758, filed Oct. 27, 2017, the entirety of which is incorporated herein by reference.

### FIELD OF THE INVENTION

This application relates generally to vaporization of phyto materials, and more specifically to method and devices for vaporizing phyto materials.

### INTRODUCTION

The following is intended to introduce the reader to the detailed description that follows and not to define or limit the claimed subject matter.

Aromatherapy generally uses essential oils for therapeutic benefits. Essential oils can be extracted from phyto materials, such as the leaves of plants. In some cases, essential oils may be massaged into the skin to provide therapeutic benefits. In other cases, essential oils may be ingested or inhaled for therapeutic purposes.

In some cases, phyto materials may be heated in order to release the essential oils therefrom. By heating phyto materials at predetermined temperatures, essential oils and extracts can be boiled off. Depending on the temperature at which the phyto materials are heated, an aroma or vapor may be given off. This vapor may be inhaled by a user for its therapeutic benefits.

Various methods of vaporizing phyto materials, such as cannabis products, are known. Devices that vaporize phyto materials are generally known as vaporizers.

### SUMMARY

The following introduction is provided to introduce the reader to the more detailed description to follow and not to limit or define any claimed or as yet unclaimed invention. One or more inventions may reside in any combination or sub-combination of the elements or process steps disclosed in any part of this document including its claims and figures.

In accordance with an aspect of this disclosure, there is provided a vaporization device for phyto material. The vaporization device can include a device body having: a heating chamber having a chamber wall defining a chamber cavity, the chamber wall including a perforated wall section; a heating element assembly positioned adjacent to the perforated wall section, the heating element assembly having an external air inlet fluidly connected to an external environment; a heating element flow path that extends from the air inlet to the chamber cavity via the perforated wall section; a control circuit electrically coupled to the heating element assembly; and an energy storage module electrically coupled to the control circuit; and a lid movably mounted to the device body, the lid movable between an open position and a closed position, the lid having: an outer wall; a lid floor having a perforated floor section; an inner lid space defined between the outer wall and the lid floor; an air cooling assembly positioned within the inner lid space at least partially overlying the perforated floor section; and an inhalation aperture defined in the outer wall, the inhalation aperture fluidly coupled to the air cooling assembly downstream from the air cooling assembly; where, in the open position, the chamber cavity is open to the external environment and the phyto material is loadable within the chamber cavity; in the closed position, the lid and the heating chamber enclose the chamber cavity, and at least a portion of the perforated floor section overlies the chamber cavity so that the chamber cavity and the inner lid space are fluidly connected; in the closed position, the heating element assembly is energizable to heat a defined region of the chamber cavity to a predetermined vaporization temperature; and the air cooling assembly is alignable with the heating element assembly to define a vapor flow path from the chamber cavity through the perforated floor to the air cooling assembly and the inhalation aperture.

In some embodiments, the heating chamber has a heating chamber length; the heating element assembly has a heating element length that is less than the heating chamber length; and the heating element assembly is moveable along the chamber length to heat the defined heating region of the chamber cavity.

In some embodiments, the air cooling assembly has a cooling element length that is less than the heating chamber length; and the air cooling assembly is moveable within the inner lid space, such that, in the closed position, the air cooling assembly is alignable with the defined heating region.

In some embodiments, in the closed position, movement of the heating element assembly and the air cooling assembly is synchronized.

In some embodiments, the chamber cavity is divided into a plurality of segments along the chamber length.

In some embodiments, the plurality of segments are separated by at least one divider.

In some embodiments, a plurality of dividers are positioned within the heating chamber at regular intervals along the chamber length.

In some embodiments, each segment defines a phyto material receiving area sized to receive a predetermined volume of phyto material; the heating element assembly is moveable along the chamber length to heat a defined segment of the chamber cavity; and the segments are separately heatable as the heating element is moved along the chamber length.

In some embodiments, the heating element assembly comprises a plurality of heaters positioned along the chamber length, each heater aligned with a respective heating region.

In some embodiments, each heater is independently energizable to heat the respective heating region of the chamber cavity aligned with that heater.

In some embodiments, the chamber cavity is rectangular.

In some embodiments, the vaporization device can include an airflow sensor fluidly coupled to the heating element flow path between the air inlet and the chamber cavity.

In some embodiments, the perforated wall section defines a base and two sidewalls of the rectangular chamber cavity; the heating element assembly includes a u-shaped heater shaped to at least partially surround the base and the two sidewalls defined by the perforated wall section.

In some embodiments, the u-shaped heater is positioned in the heating element flow path between the air inlet and the chamber cavity, and the u-shaped heater includes heating element outlets facing each of the base and the two sidewalls of the heating chamber.

In some embodiments, the heating chamber is cylindrical; and the heating element assembly includes a semi-annular heater.

In some embodiments, the device body further includes a scale coupled to the chamber cavity that is arranged to weigh phyto material loaded into the chamber cavity.

In some embodiments, the vaporization device has a first end and an opposed second end; the lid extends from the first end to the second end; the heating chamber has a heating chamber length that extends at least partially from the first end to the second end; the heating element assembly is moveable along the chamber length to heat the defined heating region of the chamber cavity; the air cooling assembly is moveable within the inner lid space; each of the heating element assembly and the air cooling assembly is moveable toward the inhalation aperture in response to a user inhaling through the inhalation aperture.

In some embodiments, the vaporization device has a first end and an opposite second end; the lid extends from the first end to the second end; the heating chamber has a heating chamber length that extends at least partially from the first end to the second end; a plurality of heaters positioned along the chamber length, each heater defining a respective heating region of the chamber cavity; and each of the heaters is individually energizable in response to a user inhaling through the inhalation aperture.

In some embodiments, the air cooling assembly may have a fixed position and may be alignable with the heating element assembly and each of the vaporization regions through an air cooling assembly air inlet. In some embodiments, the air cooling assembly air inlet may define the only vapor outlet of the chamber cavity.

In accordance with an aspect of this disclosure, there is provided a method of vaporizing phyto material using a vaporization device having a heating chamber with a heating chamber length. The method can include: loading the phyto material into a selected vaporization region of the heating chamber; enclosing the heating chamber with a lid; moving a heating element assembly along the heating chamber length to align the heating element assembly with the selected vaporization region; activating the heating element assembly; and drawing air through the heating element assembly and into the selected vaporization region.

In some embodiments, moving the heating element assembly along the chamber length includes moving an air cooling assembly along the heating chamber to align the air cooling assembly with the selected vaporization region. In some embodiments, the air cooling assembly may remain stationary and may be aligned with each of the vaporization regions through an air cooling assembly air inlet.

In some embodiments, loading the phyto material into the heating chamber comprises loading a plurality of heating chamber segments with a predetermined dose of the phyto material.

In some embodiments, loading the phyto material into the heating chamber may involve loading the phyto material into a heating chamber segment until an embedded scale identifies a predetermined dose.

In some embodiments, the method may also include moving the heating element assembly along the heating chamber length to a plurality of selected vaporization regions and sequentially activating the heating element assembly when the heating element assembly is aligned with each selected vaporization region.

In accordance with an aspect of this disclosure, there is a provided method of vaporizing phyto material using a vaporization device having a heating chamber with a heating chamber length and a plurality of heaters positioned along the heating chamber length. The method can include: loading the phyto material into a selected vaporization region of the heating chamber; enclosing the heating chamber with a lid; energizing a subset of heaters from the plurality of heaters, the subset of heaters corresponding to the selected vaporization region; drawing air through the heating element assembly and into the selected vaporization region.

In some embodiments, the method may include moving an air cooling assembly along the heating chamber to align the air cooling assembly with the selected vaporization region. In some embodiments, the air cooling assembly may remain stationary and may be aligned with each of the vaporization regions through an air cooling assembly air inlet.

In some embodiments, the method may include providing a mass airflow sensor disposed between the air inlet and the chamber cavity for measuring a mass of air drawn through the heating element assembly.

These and other aspects and features of various embodiments will be described in greater detail below.

### BRIEF DESCRIPTION OF THE DRAWINGS

For a better understanding of the described embodiments and to show more clearly how they may be carried into effect, reference will now be made, by way of example, to the accompanying drawings in which:

FIG. 1 is a perspective view of an example vaporization device with the lid in an open position in accordance with an embodiment;

FIG. 2 is a perspective view of an example lid of the vaporization device of FIG. 1 with a top surface of the lid removed in accordance with an embodiment;

FIG. 3 is a perspective view of a heating unit for the vaporization device of FIG. 1 in accordance with an embodiment;

FIG. 4 is a perspective view of a heating element assembly for the vaporization device of FIG. 1 in accordance with an embodiment;

FIG. 5 is a rear plan view of the example heating unit of FIG. 3 with an air cooling assembly positioned adjacent thereto;

FIG. 6 is a perspective view of an alternative heating unit for the vaporization device of FIG. 1 in accordance with an embodiment;

FIG. 7 is a perspective view of another alternative heating unit for the vaporization device of FIG. 1 in accordance with an embodiment;

FIG. 8 is a perspective view of another alternative heating unit for the vaporization device of FIG. 1 in accordance with an embodiment;

FIG. 9 is a perspective view of another example vaporization device with the lid in an open position in accordance with an embodiment;

FIG. 10 is a side perspective view of another example vaporization device with the lid in an open position in accordance with an embodiment;

FIG. 11 is a side perspective view of the example vaporization device of FIG. 10 with perforated side and bottom walls of the heating chamber removed;

FIG. 12 is a rear perspective view of the example vaporization device of FIG. 10;

FIG. 13 is a side plan view of the example vaporization device of FIG. 10 with the lid in a closed position;

FIG. 14 is a partial cutaway view of the example vaporization device of FIG. 10;

FIG. 15 is a perspective view of an example air flow sensor that may be used with the vaporization device of FIG. 10 in accordance with an embodiment;

FIG. 16 is a perspective view of an alternative heating unit in accordance with an embodiment;

FIG. 17 is a perspective view of the heating unit of FIG. 16 with an air cooling assembly separated from the heating element assembly;

FIG. 18 is a perspective view of an air cooling assembly and heating element assembly for the heating unit of FIG. 16 with a housing layer removed in accordance with an embodiment;

FIG. 19 is a perspective view of an example heating chamber for the heating unit of FIG. 16 in an open position;

FIG. 20 is a perspective view of the heating unit of FIG. 16 along with a control circuit and energy storage module in accordance with an embodiment; and

FIG. 21 is a perspective view of an alternative heating unit in accordance with an embodiment.

The drawings included herewith are for illustrating various examples of articles, methods, and apparatuses of the teaching of the present specification and are not intended to limit the scope of what is taught in any way.

### DETAILED DESCRIPTION

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising,” and variations thereof mean “including but not limited to,” unless expressly specified otherwise. A listing of items does not imply that any or all of the items are mutually exclusive, unless expressly specified otherwise. The terms “a,” “an,” and “the” mean “one or more,” unless expressly specified otherwise.

Embodiments described herein relate generally to vaporization of phyto material and phyto material products. Phyto material products may be derived from phyto materials such as the leaves or buds of cannabis plants.

Phyto material is often vaporized by heating the phyto material to a predetermined vaporization temperature. The emitted phyto material vapor can then be inhaled by a user for therapeutic purposes. The vapor may often be emitted at a temperature that can be uncomfortable for a user to inhale. Accordingly, it may be desirable to cool the vapor prior to inhalation.

It may also be desirable to control the volume of vapor inhaled by a user. Users may not be aware of the quantity of vapor being inhaled in a given vaporization session. This may result in undesirable side effects if more phyto material than desired is vaporized. Controlling the dose of phyto material that is inhaled by a user may reduce or avoid these unwanted side effects.

Embodiments described herein related generally to methods and devices for vaporizing phyto material, in particular loose-leaf phyto material.

In embodiments described herein, a vaporization device may include a device body and a lid movably mounted to the device body. The device body can include a heating unit that has a heating chamber and a heating element assembly. The heating element assembly can be positioned adjacent to the heating chamber. The heating element assembly can be used to heat the heating chamber.

The heating chamber may have a heating chamber length extending along a majority of the length of the device body. The heating element assembly may be configured to selectively heat specific regions within the heating chamber. Users may control a volume of phyto material vapor inhaled by controlling the regions that are heated during a given vaporization session.

In some cases, the heating element assembly may be moveable along the heating chamber length to heat defined regions of the heating chamber. Alternatively, the heating element assembly may include a plurality of region-specific heating elements positioned along the heating chamber length. Each region-specific heating element can be used to heat the defined region of the heating chamber.

The heating chamber may be separated into a plurality of chamber segments along the heating chamber length. The chamber segments can be physically separated by dividers.

Phyto material may be separately loadable into each heating chamber segment. Each heating chamber segment may also be heatable separately and independently by the heating element assembly.

In some cases, dividers may be positioned at regular intervals along the heating chamber length. This may provide the heating chamber with a plurality of evenly sized heating segments.

Alternatively, the dividers may be positioned at different intervals along the heating chamber length. This may allow the vaporization device to include heating chamber segments that are sized to receive different volumes of phyto material. This may allow a user to select different doses of phyto material by selecting and activating heating chamber segments of different sizes.

The lid can be moved between an open position and a closed position. The lid may have an outer lid wall or walls and an inner lid floor. When the lid is closed, the inner lid floor may face the heating chamber. The inner lid space may be fluidly connected to the heating chamber. The inner lid floor can include one or more apertures that define a vapor outlet for the heating chamber. For example, the inner lid floor can be perforated.

An inhalation aperture may be defined in the outer lid wall. The inhalation aperture may be fluidly connected to the heating chamber to receive phyto material vapor emitted from the heating chamber.

The lid can define an inner lid space or inner lid volume between the outer lid walls and the inner lid floor. An air cooling assembly may be positioned within the inner lid space. The air cooling assembly can be fluidly connected between the heating chamber and the inhalation aperture. The air cooling assembly may cool the vapor emitted from the heating chamber prior to the vapor reaching the inhalation aperture. This may allow a user to inhale vapor at a lower temperature.

When the lid is in the open position, the chamber cavity may be open to the external environment. A user may then load phyto material into a chamber cavity within the heating chamber. When the lid is closed, the lid floor and the heating chamber walls can enclose the chamber cavity. At least a portion of the perforated lid floor can cover the chamber cavity to provide fluid communication between the chamber cavity and the inner lid space.

In some cases, the air cooling assembly may be moveable within the inner lid space. For example, the air cooling assembly may be moveable along the length of the heating chamber. This may allow the air cooling assembly to be aligned with the heating segment that is being vaporized.

In some cases, the air cooling assembly may be stationary within the inner lid space. The air cooling assembly may then be aligned with the vapor outlet of the heating chamber. This may ensure that vapor emitted from phyto material within the heating chamber passes through the air cooling assembly prior to reaching the inhalation aperture.

With the lid in the closed position, the heating element assembly can be energized to heat the heating chamber. Phyto material in the chamber cavity can be heated to a predetermined temperature and vaporized. The heating element assembly may have a plurality of air input ports fluidly connected to the external environment. The walls of the heating chamber may include air inlets or perforations to allow air to be drawn through the heating element assembly and into the heating chamber.

When a user inhales from the inhalation aperture, ambient air may be drawn from the external environment into the chamber cavity through the plurality of air input ports and the perforated wall. In the chamber cavity, the ambient air can mix with the vaporized phyto material while being drawn by the user's inhalation through the air cooling assembly and exiting the inhalation aperture.

In some embodiments, movement of the heating element assembly and the air cooling assembly may be synchronized. For example, the heating element and air cooling assembly may be mechanically connected to ensure synchronized movement. Alternatively, movement of the heating element assembly and air cooling assembly may be synchronized by a control circuit included in the device body.

Alternatively, in embodiments in which a plurality of heating elements are positioned along the length of the heating chamber, movement of the air cooling assembly may be controlled to align the air cooling assembly with the heating element or elements to be energized.

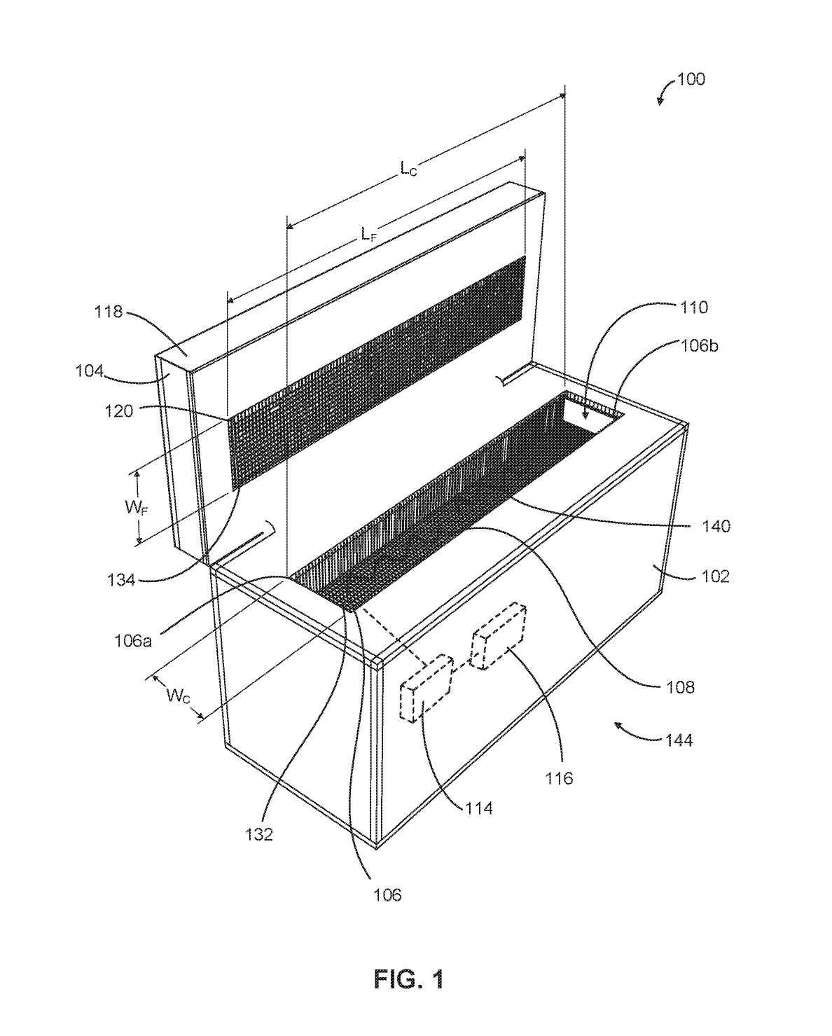

Referring now to FIGS. 1-5, shown therein is an example of a vaporization device 100. Vaporization device 100 is an example of a vaporization device usable to vaporize phyto material. Vaporization device may be used to vaporize loose and/or ground phyto material.

FIG. 1 shows a perspective view of the vaporization device 100. Vaporization device 100 includes a device body 102 and a lid 104. Lid 104 can be moveably mounted to device body 102.

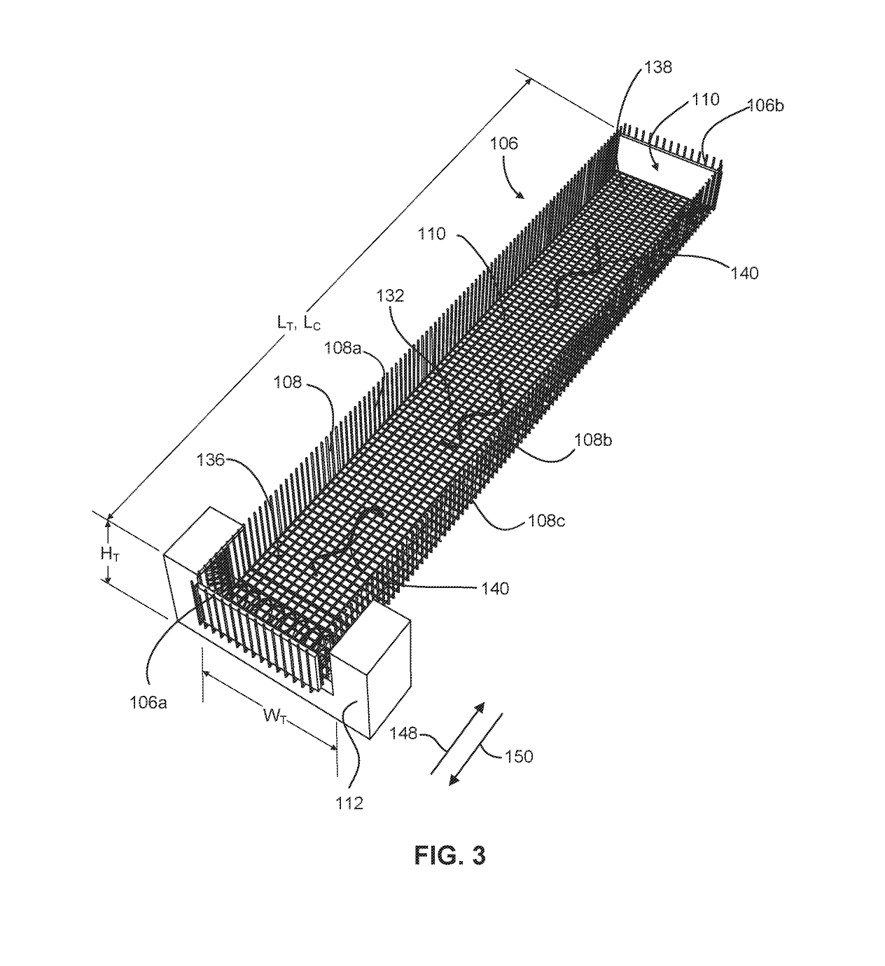

The device body 102 has a heating unit that includes a heating chamber 106 and a heating element assembly 112. The heating element assembly 112 can be positioned adjacent to the heating chamber 106 (see e.g. FIG. 3). The heating element assembly 112 can be used to heat one or more defined regions of the heating chamber 106.

The heating chamber 106 extends from a first end 106a to a second end 106b along a chamber length L_{c}. The heating chamber 106 can include a first end wall at the first end 106a, a second end wall at the second end 106b. The heating chamber 106 can also include one or more sidewalls extending from the first end wall to the second end wall. The sidewalls can define a chamber cavity 110 of the heating chamber 106. Phyto material may be loaded to the chamber cavity 110 in preparation for vaporization.

Some or all of the sidewalls of the heating chamber 106 may be perforated. This may allow air to flow into the chamber cavity 110 of heating chamber 106.

In the example shown, heating chamber 106 is rectangular. Heating chamber 106 includes a pair of sidewalls 108a and 108b extending from the first end wall to the second end wall. Heating chamber 106 also includes a third sidewall 108c extending from the first end wall to the second end wall that may also be referred to as a base or floor. The sidewalls 108a and 108b of heating chamber 106 (including the base sidewall 108c) may each be perforated.

Heating chamber 106 also has an open upper end or side. Phyto material can be loaded into the chamber cavity 110 through this open upper end.

The lid 104 may be moved between an open position (shown in FIG. 1) and a closed position (not shown). In the open position, the upper end of the chamber cavity 110 can be exposed. This may allow a user to load phyto material for vaporization and/or dispose of vaporized phyto material. When the lid 104 is moved to the closed position, the lid 104 and the heating chamber 106 can enclose the chamber cavity 110.

The heating element assembly 112 can be positioned at least partially surrounding the exterior of the heating chamber 106. The heating element assembly 112 can be energized to emit heat. The heat from the heating element assembly 112 can heat the chamber cavity 110, and in turn the phyto material positioned in the chamber cavity 110.

In some embodiments, the heating element assembly 112 may include one or more resistive heating elements. In the example shown, the heating element assembly 112 includes a coil heating element 115. The coil heating element 115 may be activated by directing current through the coil 115. The coil 115 may then emit heat. Heat from the heating element assembly 112 can radiate into the heating chamber 106 to heat phyto material in the chamber cavity 110 to a predetermined vaporization temperature. Phyto material vapor can then be emitted.

Phyto material may be positioned in the chamber cavity 110 at different regions along the length of the heating chamber 106. The heating element assembly 112 may be operable to selectively heat different regions of the chamber cavity 110. This may allow the phyto material within a given region to be vaporized without vaporizing phyto material in a different region of the chamber cavity 110.

Vaporization device 100 may include an energy storage module 116 such as a battery electrically coupled to heating element assembly 112. Energy storage module 116 may be used to energize heating element assembly 112 to heat the phyto material within the chamber cavity 110.

The vaporization device 100 may include a control circuit 114 electrically coupled to the heating element assembly 112 and/or energy storage module 116. The control circuit 114 can control the operation of the heating element assembly 112. The control circuit 114 may be used to activate/deactivate the heating element assembly 112. The control circuit 114 may be used selectively activate the heating element assembly 112 to heat only selected regions of the chamber cavity 110.

The control circuit 114 may also be used to adjust the settings of vaporization device 100, such as a predetermined vaporization temperature. The control circuit 114 may control the flow of current through the heating element assembly 112 in accordance with a selected vaporization temperature.

The control circuit 114 may also manage the operation of other components of vaporization device 100 such as user input and/or user feedback components. For instance, vaporization device 100 may include one or more output components that provide visual or audible signals to a user regarding the configuration and settings of vaporization device 100. In some cases, vaporization device 100 may include wireless communication modules to allow the vaporization device 100 to communicate with another wireless device such as a smartphone or tablet.

Energy storage module 116 may be a rechargeable energy storage module, such as a battery or super-capacitor. Vaporization device 100 may include a power supply port (e.g. a USB-port or magnetic charging port) that allows the energy storage module 116 to be recharged. The energy storage module 116 may optionally be removable to allow it to be replaced.

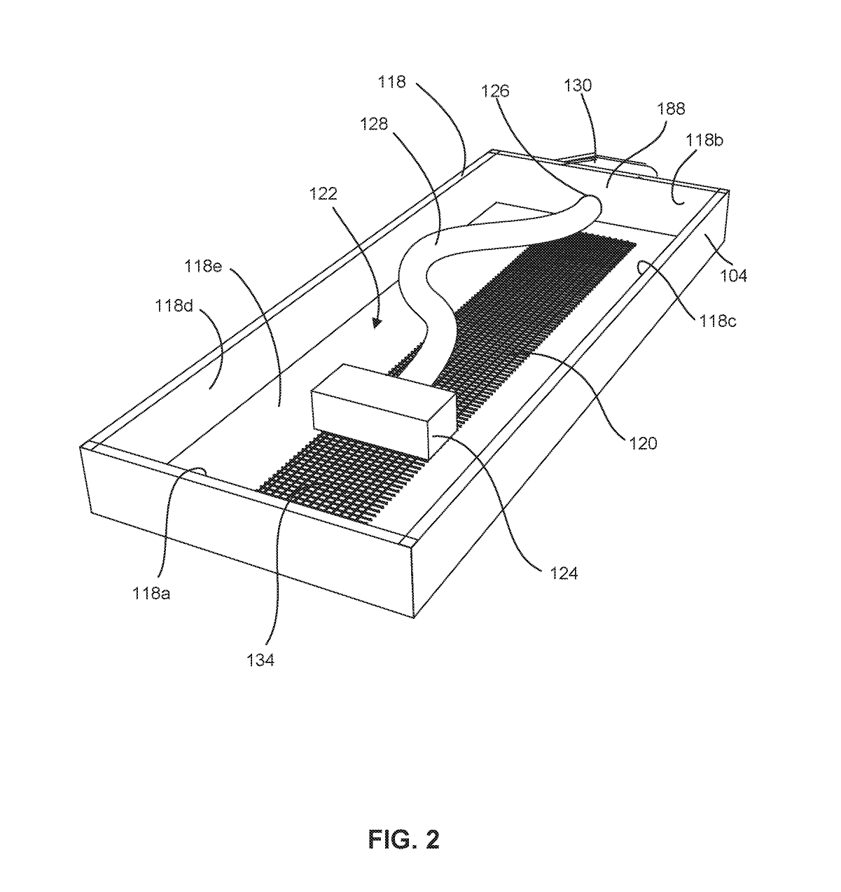

FIG. 2 shows a perspective view of the lid 104 with a top wall of the lid 104 removed. The lid includes a first end wall 118a, a second end wall 118b, sidewalls 118c and 118d extending from the first end wall 118a to the second end wall 118b, and a floor 118e extending from the first end wall 118a to the second end wall 118b and between the sidewalls 118c and 118d. An inhalation aperture 130 can be defined in the outer wall of the lid 104, for instance in the second end wall 118b as shown.

In some embodiments, the inhalation aperture 130 may include a mouthpiece that extends outward from the wall of lid 104, e.g. as shown. Alternatively, the inhalation aperture 130 may be flush with the wall of lid 104. Optionally, the mouthpiece of inhalation aperture 130 may be removable to allow the mouthpiece to be cleaned and/or replaced.

A section 120 of the inner lid floor 118e can be perforated. The perforated floor section 120 may be shaped to be aligned with, or substantially match, the open upper end of the chamber cavity 110. For example, as shown in FIG. 1, a floor length L_{F} and a floor width W_{F} of the perforated floor section 120 can be sized to substantially match the chamber length L_{C} and chamber width W_{C} of the heating chamber 106, respectfully. In some cases, the perforated section may cover the entirety of the inner lid floor 118e.

An inner lid space 122 is defined between the walls 118 of the lid 104 and the top wall (not shown). An air cooling assembly 124 can be positioned within the inner lid space 122. The air cooling assembly 124 can include an air inlet that is positioned to face the perforated floor section 120. When the lid 104 is in the closed position, the air cooling assembly 124 can be fluidly connected to the chamber cavity 110 through the perforated floor section 120.

The air cooling assembly 124 can include an air outlet 126 that is fluidly connected to the inhalation aperture 130. The air cooling assembly 124 can include a fluid conduit extending from the air inlet to the air outlet 126 so that air drawn into the air cooling assembly 124 flows downstream to the inhalation aperture 130. The inhalation aperture 130 may be connected to the air outlet 126 solely, or rather, the air outlet 126 may provide the sole fluid connection between the inhalation aperture 130 and the heating chamber 108. This may ensure that air from within the vaporization device 100 can only enter the inhalation aperture 130 through the air cooling assembly 124. The air cooling assembly fluid conduit may be provided as a flexible hose 128 that fluidly connects the air inlet and the air outlet 126.

Vapor from the chamber cavity 110 may enter the air inlet of the air cooling assembly 124 at a first temperature T₁ and exit through air outlet 126 at a second temperature T₂ that is lower than the first temperature T₁. This may provide a user with a more comfortable, and safer, temperature of vapor for inhalation.

In some embodiments, the air cooling assembly 124 can contain a fan (not shown) that cools air passing within. In some embodiments, the air cooling assembly 124 can contain a refrigerant, such as water vapor, (not shown) that can be released to cool air as it passes. For example a TEC (thermoelectric cooler) can be used with a heatsink assembly to cool the air passing within. In some cases, the ambient air within the inner lid space 122 may simply cool air flowing through the conduit of the air cooling assembly 124.

In some embodiments, the heating assembly 112 may be moveable along the length of the heating chamber 106. This may allow the heating assembly 112 to be positioned to heat a defined region of the chamber cavity 110. In the illustrated example, the heating element assembly 112 may be positioned anywhere along the chamber length L_{C} to direct heat into at least a portion of the chamber cavity 110. The control circuit 114 may control the position of the heating element assembly 112 to ensure that heating element assembly is aligned with a selected vaporization region. Optionally, the control circuit 114 can be used to control an actuator (not shown) that controllably moves the heating element assembly 112 along the length of the heating chamber 106.

FIG. 3 shows a perspective view of the heating chamber 106 of FIG. 1 with the heating element assembly 112 adjacent to a region of the heating chamber 106. As shown in FIG. 3, the heating element assembly 122 surrounds only a portion of the chamber cavity 110. The heating element assembly 122 may then be moved along the length of the heating chamber 106 to heat other regions of the chamber cavity 110.

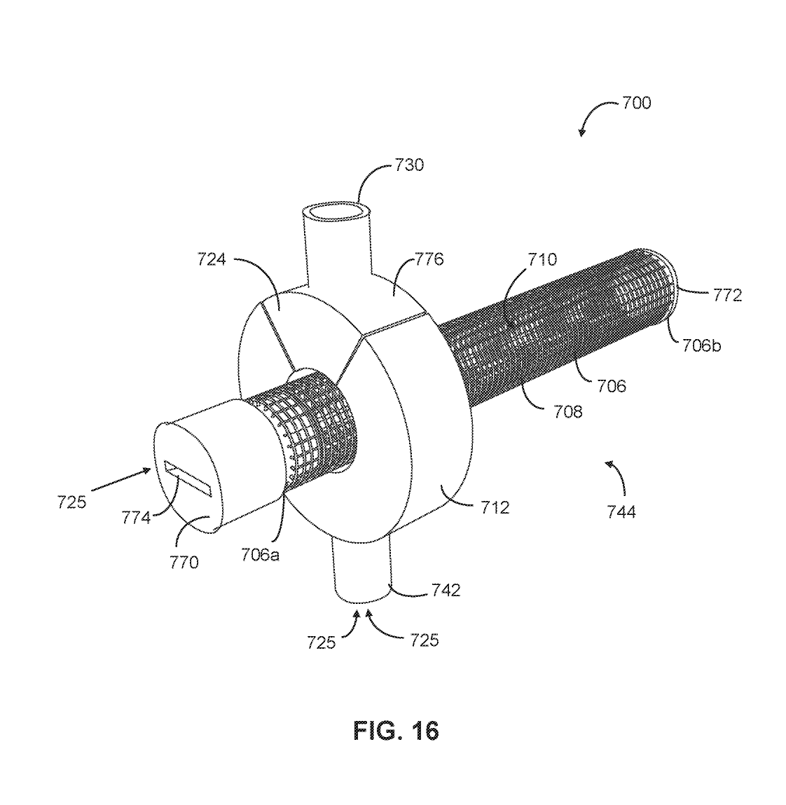

In the example shown, the heating chamber 106 is substantially rectangular. Other shapes and configurations may be used, such as a cylindrical heating chamber (see e.g. FIG. 16 described herein below). Typically, however, the heating chamber 106 can have one dimension that is greater than its other dimensions. This may allow a heating element to translate along that longer dimension to heat different regions of the heating chamber or allow multiple heating elements to be positioned along the longer dimension.

The heating chamber 106 may define a chamber cavity 110 that has a rectangular shape similar to a trough 106. The chamber cavity 110 has a trough length L_{T}, a trough height H_{T} and a trough width W_{T}. The trough length L_{T} is greater than the trough width W_{T}. The trough length L_{T} is also greater than the trough height H_{T}. As shown, the trough 106 has a first region 136 at a first end of the chamber cavity 110 and a last region 138 at the opposite end of the chamber cavity 110.

The chamber cavity 110 generally defines a volume within which a user may add phyto material. For example, a user may add loose leaf phyto material 140 within the chamber cavity through the trough length L_{T}. In some cases, phyto material may be substantially evenly distributed throughout the chamber cavity 110. Alternatively, one or more regions may be loaded with a different quantity of the loose leaf phyto material 140. A user may then vaporize the phyto material in the different regions of the chamber cavity 110 by moving the heating element assembly 112 along the trough length.

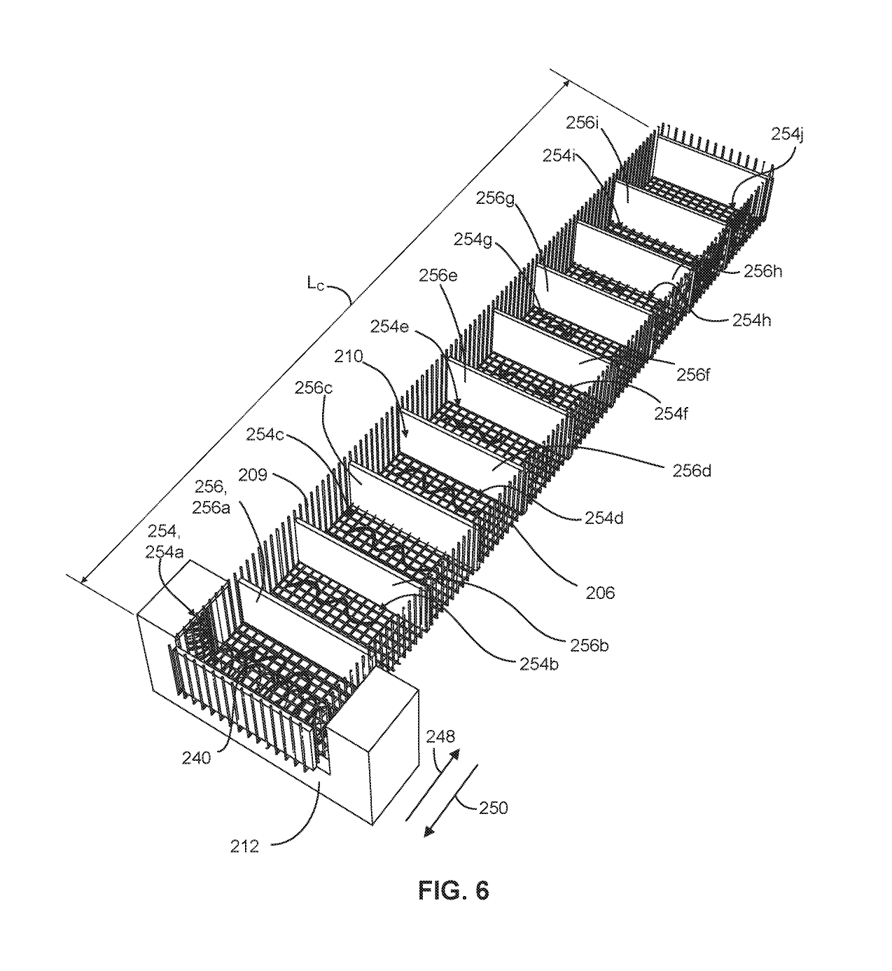

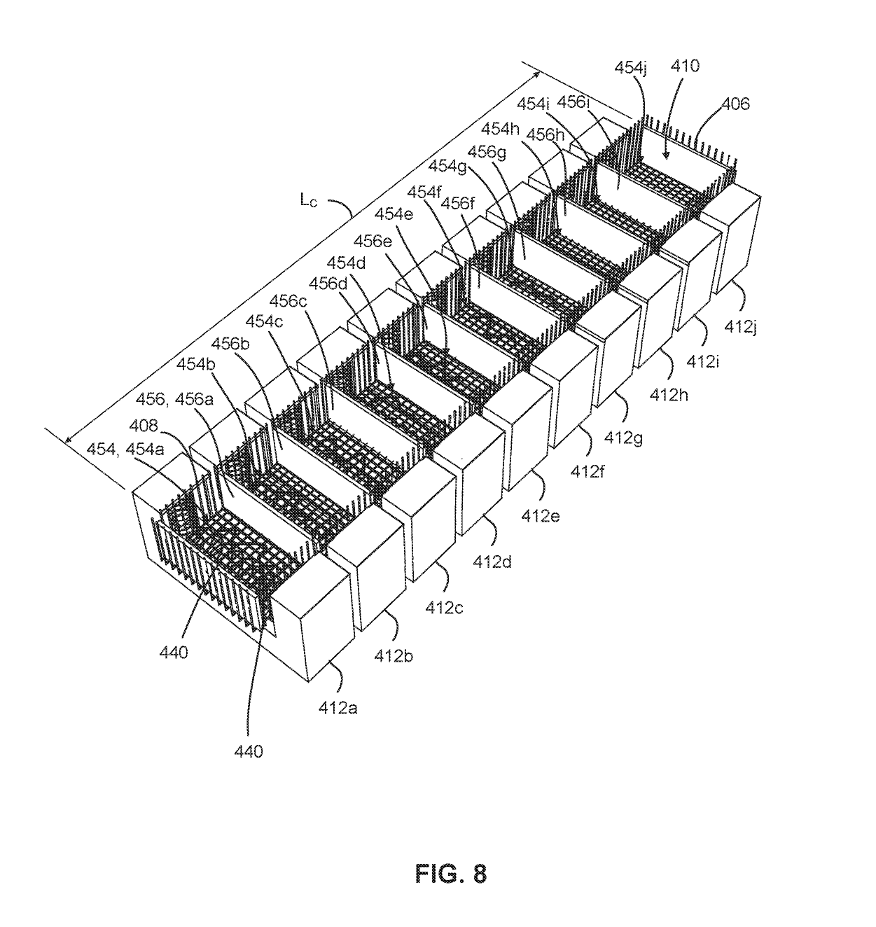

In some embodiments, the regions of the chamber cavity 110 may be separated using dividers (see e.g. FIGS. 6 and 8 described herein below). This may provide a user with more control over the volume of phyto material positioned within each region.

Although vaporization device 100 has been described in the context of the vaporization of loose leaf or ground phyto material 140, various forms of phyto material products may be used with vaporization device 100. For example, phyto material may be packed into a pre-compressed tablet (not shown). The pre-compressed tablet may have a predetermined dose and can be loaded directly within the chamber cavity 110 of the heating chamber 106.

The heating element assembly 112 may be shaped to partially surround or envelope the heating chamber 106. In the example shown, the heating element assembly 112 is substantially U-shaped to correspond to the rectangular shaped of the heating chamber 106. By virtue of its shape, the U-shaped heating element assembly 112 can direct heat into the heating chamber 106 from three sides (i.e. sidewalls 108a and 108b and base sidewall 108c). In the closed position, the perforated floor surface 120 of the lid 104 can form a fourth side that encloses the phyto material within chamber cavity 110 of the heating chamber 106. In other embodiments, the shape of the heating element assembly 112 may be modified to accommodate different shapes of heating chambers 106.

The inner dimensions of the heating element assembly 112 may be defined to correspond to the outer dimensions of the heating chamber 106. For example, the inner walls of the heating element assembly 124 may define a groove 146 within which the heating chamber 106 may sit. Groove 146 has a groove length L_{G}, a groove width W_{G} and a groove height H_{G}. The groove height H_{G} and the groove length L_{G} may be substantially equal to, although slightly greater than, the chamber height H_{T} and the chamber width W_{T}, respectively. The grove width W_{G} is shorter than the trough length L_{T}. In this way, as shown in FIG. 3, when the trough 106 is positioned within the groove 146, the heating element assembly 112 can be moved along the trough length L_{T} in both a forward direction 148 and a rearward direction 150.

In some embodiments, the heating element assembly 112 can be moved along the chamber length L_{C} to heat a specific region (e.g. first region 136, last region 138, etc.) of the chamber cavity 110. In some embodiments, each region can be loaded with a predetermined dose of the loose leaf phyto material 140. In this way, each region can be heated individually, and in some cases sequentially, to vaporize one predetermined dose at a time.

Vaporing loose phyto material 140 by regions may facilitate providing a controlled dose, even where the user fills the trough 106 with the loose leaf phyto material 140 (after sufficiently grinding the phyto material) and distributes it as desired within the chamber cavity 110.

In some embodiments, the heating element assembly 112 may be moved using an actuator such as an electric motor or a drive screw (not shown). The actuator may be controlled by the control circuit 114. In some embodiments, a user may be able to manually adjust the position of heating element assembly by activating the actuator (e.g. turning drive screw clockwise or counterclockwise). In another example, the heating element assembly 112 can be moved by a spring-loaded slider (not shown). A trigger, dial, or pull-pin may be used to adjust the tension in the spring to move the heating element assembly 112 along the chamber length L_{C}.

In some embodiments, the air cooling assembly 124 can be moved within the inner lid space 122. In this way, in the closed position, the air cooling assembly 124 may be aligned with the region of the chamber cavity 106 that is currently aligned with the heating element assembly 112. The air cooling assembly 124 can be moved in a manner similar to the heating assembly 112 e.g. using a motor, a spring-loaded slider or a drive screw for example.

In some embodiments, movement of the heating element assembly 112 and the air cooling assembly 124 may be synchronized (i.e. they may move together at the same time). This may ensure that vapor emitted from the phyto material is drawn directly into the air cooling assembly 124 while minimally contacting other regions, or without passing through other regions of the chamber cavity 110.

In some embodiments, the air cooling assembly 124 may be stationary. In some such embodiments, the perforated section of the inner lid floor 118e may be more contained. For example, in some embodiments in which the air cooling assembly 124 is stationary, the inner lid floor 118e may only include a perforated section that aligns with the inlet of the air cooling assembly 124. In some cases, the heating element assembly 112 may still be moveable even though the air cooling assembly 124 remains stationary.

In some embodiments, the vaporization device 100 may be configured to vaporize phyto material in chamber regions in a predefined sequence. For example, the heating element assembly 112 may be controlled to vaporize chamber regions beginning from the first region 136 and moving sequentially along the length of the chamber cavity 110 to the last region 138.

In some embodiments, the heating element assembly 112 and the air cooling assembly 124 can be drawn toward the inhalation aperture 130 by the suction force generated by a user inhaling through the inhalation aperture 130. This may allow the user to continue vaporizing phyto material in regions of the chamber cavity 110 that have not yet been vaporized. In some cases, this may allow a user to vaporize phyto material in multiple regions of the chamber cavity 110 in a single inhalation.

For example, the heating element assembly 112 and the air cooling assembly 124 may initially be positioned at the first end 136 of the heating chamber 106. The suction force may draw the heating element assembly 112 and/or the air cooling assembly 124 in the forward direction 148 toward the inhalation aperture 130. In some cases, heating element assembly 112 and/or air cooling assembly 124 may be drawn in the forward direction without an active device driving movement of the heating element assembly and/or the air cooling assembly 124. In some cases, the suction force may be aided by an active device driving movement of the heating element assembly 112 and/or the air cooling assembly 124 in the forward direction 148 toward the inhalation aperture 130.

The suction force generated will depend on the force of an individual user's inhalation. That is, the rate at which the heating element assembly 112 and/or the air cooling assembly 124 are drawn in the forward direction 148 can vary based on the user's inhalation (for e.g., strength, duration and speed). This may allow the user to control their dose by controlling how they inhale through inhalation aperture 130.

In some cases, by blowing air into the inhalation aperture 130, the user may move the heating element assembly and/or the air cooling assembly 124 in the rearward direction 150 toward the first end 136. This may allow the user to vaporize any remaining phyto material that was not vaporized by a previous pass of the heating element assembly 112. In addition, it may allow the user to reset the position of the heating element assembly 112 and the air cooling assembly 124 at the first end 136 of the heating chamber 106 for a subsequent vaporization. In some cases, a user may manually re-position the heating element assembly 112 and/or air cooling assembly 124, e.g. using a tab or drive element such as a drive screw.

In some embodiments, the heating element assembly may be configured to heat the phyto material to a predetermined vaporization temperature. The predetermined vaporization temperature may vary depending on user preference and/or the form of the phyto material. For example, loose leaf phyto material may be vaporized at a predetermined vaporization temperature in a range between about 350 degrees Fahrenheit and about 450 degrees Fahrenheit. Phyto material extracts and oils on the other hand may be vaporized at temperatures ranging between about 500 and 800 degrees Fahrenheit. A user may be able to adjust the predetermined vaporization temperature using input controls. The control circuit 114 may then control the current through the heating element to adjust the vaporization temperature.

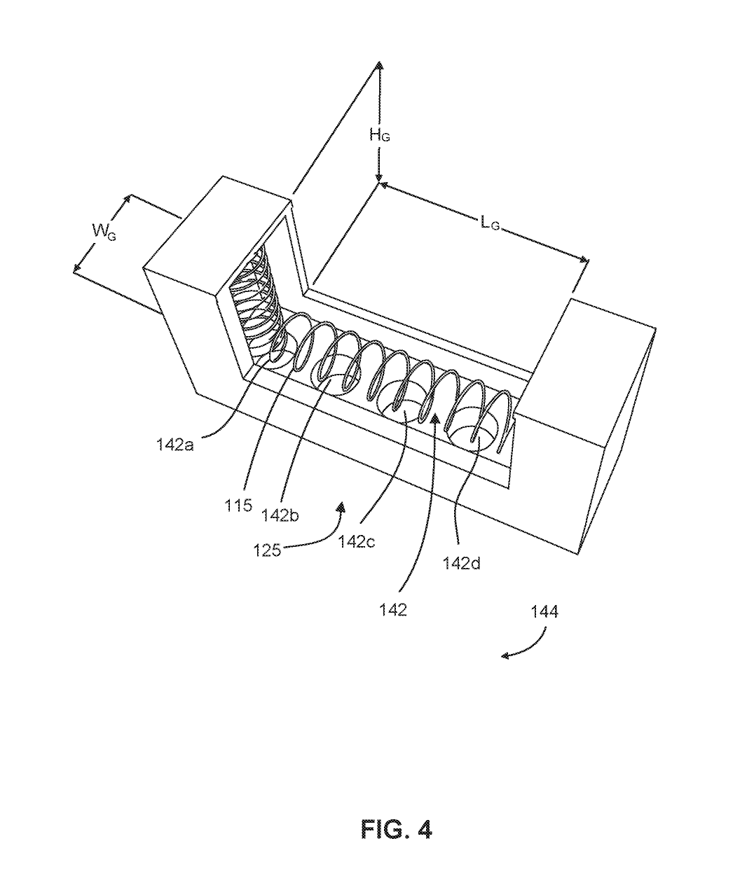

The heating element assembly 112 may also include an inner recess. The recess may be used to hold the heating element that is used to heat chamber cavity 110. In some embodiments, the heating element assembly 112 may include a resistive heating element such as a coil (e.g., coil 115). This resistive heating element may be positioned within the heating element recess (see e.g. FIG. 4). In some embodiments, the heating element assembly may include a convection heating element. Air drawn through the heating element assembly 112 can be heated by the heating element, and this heated air can vaporize phyto material positioned in the chamber cavity 110.

As discussed above, the heating chamber 106 can include perforated walls 108 around at least a portion of the chamber cavity 110. The perforated walls can include aperture or pores 132 throughout its surface. The pores 132 may permit air to pass into the chamber cavity 110. This may allow heated air from the heating element assembly 112 to pass into the chamber cavity 110 and vaporize phyto material therewithin.

The size of the apertures or pores 132 may vary depending on the form of the phyto material to be vaporized. An optimal pore size may depend on the fineness of the phyto material loaded into the chamber cavity 110 (i.e. the finer the grind, the smaller the pores 132). Smaller pores 132 may inhibit non-vaporized pieces of the phyto material from falling through the heating chamber 106 and potentially clogging the heating element. In some cases, the walls 108 of the chamber cavity 110 may be replaceable to allow the pore size to be modified.

In some embodiments, the pores 132 may be between 0.01 and 0.6 mm. For example, the pores 132 may be between 0.025 and 0.3 mm. In some embodiments, the pores 132 may be between 0.05 and 0.2 mm.

Referring back to FIG. 2, the perforated floor section 120 of lid 104 also includes apertures or pores 134 throughout its surface. The pores 134 may permit vapor to pass from the chamber cavity 110 to the inner lid space 122. The size of pores 134 may be selected to inhibit non-vaporized pieces or flakes of the phyto material from passing into the air cooling assembly 124 and out the inhalation aperture 130 into the user's mouth. Thus, the pores 134 may also provide a filtering action.

As with pores 132, the size of pores 134 may depend on the form of the phyto material being used. In some embodiments, the pores 134 may be between 0.1 and 0.6 mm. For example, the pores 134 may be between 0.025 and 0.3 mm. In some embodiments, the pores 134 may be between 0.05 and 0.2 mm. In some embodiments, the pores 132 and 134 may be substantially equal in size.

The heating element assembly 112 may have an air inlet that may include a plurality of air input ports 142. Each air input port 142 may be fluidly connected to the external environment, indicated generally as 144. In the illustrated example, heating element assembly has four air input ports, 142a, 142b, 142c, and 142d. It will be appreciated that many other configurations of the plurality air input ports 142 are possible.

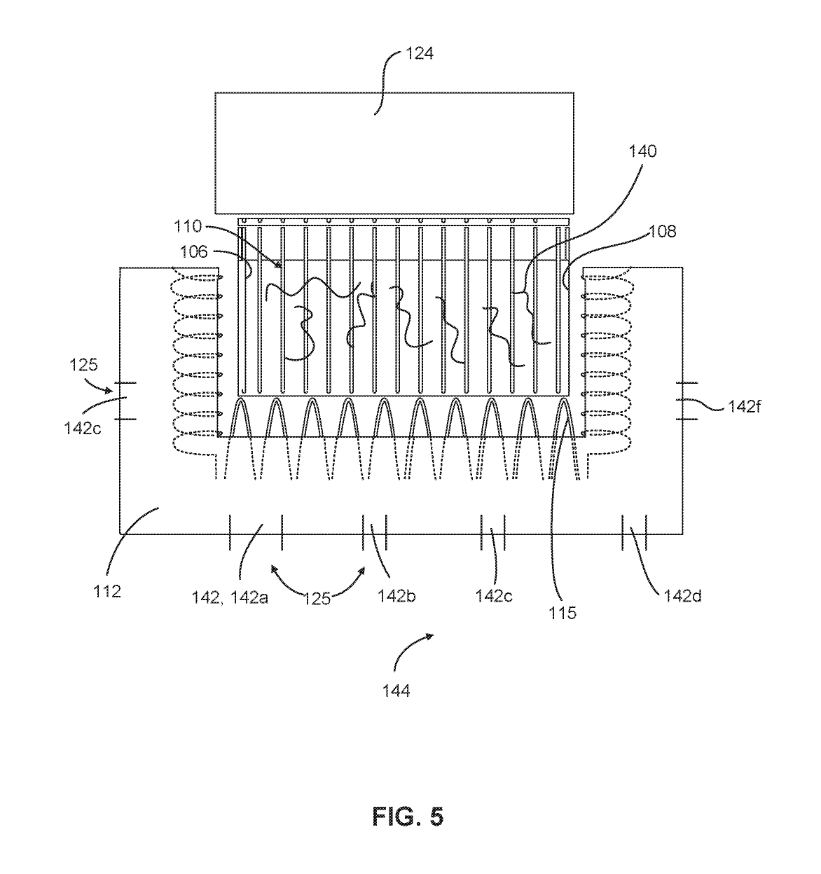

For example, as shown in FIG. 5, the heating element assembly 112 includes two additional input ports 142e and 142f defined along the sides of the U-shaped heating element assembly 112. The plurality of input ports 142 defined in the heating element assembly 112 can determine how ambient air, indicated generally as 125, drawn from the external environment 144 is passed into the chamber cavity 110 via pores 132.

Referring back to FIG. 4, heat can be emitted from the groove 146 directly into the trough 106 via the perforated wall 108 (FIG. 3). In this way, as much heat as possible can be directed (i.e. steered) into the trough 106 to heat the phyto material positioned therein. This can increase the efficiency of the vaporization device 100.

In the open position (shown in FIG. 1), the chamber cavity 110 is open to the external environment 144 and the phyto material 140 may be loaded into the chamber cavity 110 of the heating chamber 106. As discussed above, loose leaf phyto material 140 can be distributed within the chamber cavity 110 of the trough 106 (i.e. the heating chamber 106) in many possible ways.

In the closed position (not shown), the lid 104 and device body 102 may enclose the chamber cavity 110. In the closed position, at least a portion of the perforated floor 120 covers the chamber cavity 110. In this position, the chamber cavity 110 and the inner lid space 122 are in fluid communication via the pores 134.

Further, when in the closed position, the heating element assembly 112 may be energized to heat the phyto material 140 in the chamber cavity 110 to a predetermined temperature to vaporize the phyto material 140. When a user inhales from the inhalation aperture, ambient air 125 (FIGS. 4 and 5) can be drawn from the external environment 144 into the chamber cavity 110 through the plurality of air input ports 142 and the perforated wall 108. While in the chamber cavity 110, ambient air is mixed with the vaporized phyto material and is then drawn by the inhalation through the air cooling assembly 124 and out the inhalation aperture 130. The ambient air 125 drawn in via the plurality of air input ports 142 can be used to provide convection heating of the loose leaf phyto material 140 after being heated by heating assembly 112.

In the example embodiment, the lid 104 may be movably mounted to the device body 102 by a hinged connection 152. In other embodiments, the lid 104 may be movably mounted to the device body 102 by a slide-in groove connection (not shown). For example, the lid 104 may be slid on and off the body device 102 via a groove on the body device 102. In yet another embodiment (not shown), the lid 102 may be movably mounted to the device body 102 by a friction fit connection. For example, the device body 102 may have a lip around an outer edge. The lid 104 may be sized to fit within the lip and may be held in place by friction along the lip's edge. The lid 104 may contain an indent or a tab to enable the user to remove the lid 104.

Referring now to FIG. 6, shown therein is another example of a heating unit that may be used with vaporization device 100. The heating unit shown in FIG. 6 is similar to the heating unit shown in FIG. 3, except that the heating chamber 206 includes a plurality of dividers 256 positioned within the chamber cavity 210. Elements having similar structure and/or performing similar function as those in the example vaporization device 100 in FIGS. 1-5 are numbered similarly, with the reference numerals incremented by 100.

The heating chamber 206 is separated into a plurality of divided segments 254 along the chamber length C_{L}. Each divider 256 may be positioned along the chamber length C_{L} to define the size of adjacent segments 254. In the illustrated example, nine dividers 256a to 256i are shown dividing the heating chamber 210 into ten segments 254a to 254j.

In the example shown, the dividers 256 are positioned within the chamber cavity 206 at regular intervals. As a result, the segments 254 are all of substantially the same size. In some cases, the dividers 256 may be positioned to provide segments 254 appropriate for loading approximately 0.1 grams of phyto material 240 into each segment 254.

In alternative embodiments, the number and/or position of dividers 256 may be altered so that the segments 254 may vary in size. This may allow a user to easily define varying dose sizes within vaporization device 100. For example, dividers 256b, 256e, and 256h may be omitted to provide four segments of the same size shown in FIG. 6 and three segments that are twice as large.

The dividers 256 may improve vaporization of regions within the chamber cavity 210. The dividers 256 may prevent the flavor of previously vaporized phyto material from passing across segments 254. The dividers 256 may also assist in establishing a more localized vaporization of the phyto material and may reduce vaporization of the phyto material 240 in adjacent segments 254. For example, if the heating element assembly 212 is aligned with the segment 254c, the dividers 256b and 256c can prevent the phyto material 240 in the segments 245b and 254d from being vaporized.

Furthermore, by portioning the chamber cavity 210 into small segments 254, dividers 256 can allow the user to better visualize the quantity of phyto material 240 being loaded into the chamber cavity 210 for a given vaporizing session. Loading doses of the phyto material 240 in this manner can minimize inefficiencies associated with loading more phyto material 240 than necessary.

Optionally, the dividers may be removable from chamber cavity 210. This may allow a user to define the segments of the chamber cavity 210 as desired. In some cases, the inner sidewalls of the heating chamber 208 may include recesses or grooves positioned at regular intervals along the length of the chamber cavity 210. This may allow a user to easily re-position the dividers within the chamber cavity 210.

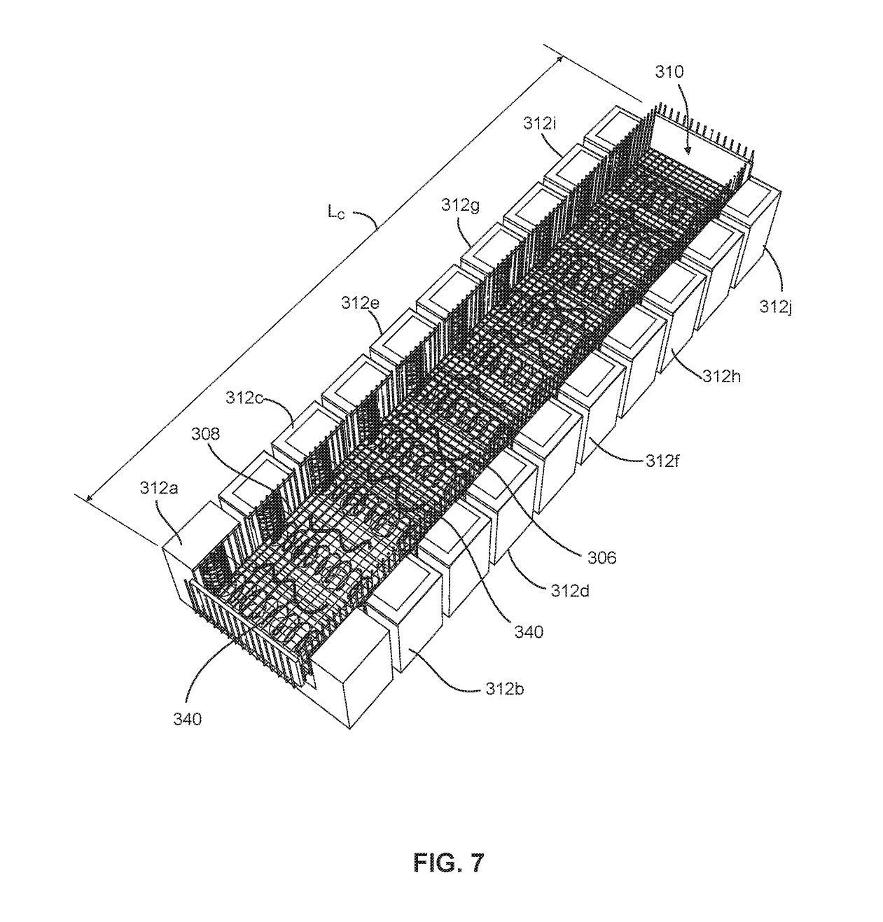

Referring now to FIG. 7, shown therein is another example of a heating unit that may be used with vaporization device 100. The heating unit shown in FIG. 6 is similar to the heating unit shown in FIG. 3, except that a plurality of heating element assemblies 312a-312j are positioned along the chamber length C_{L}. Elements having similar structure and/or performing similar function as those in the example vaporization device 100 in FIGS. 1-5 are numbered similarly, with the reference numerals incremented by 200.

Each heating element assembly 312 may be aligned with a respective region of the heating chamber 306. Each heating element assembly 312 may then be energized individual, and in some cases sequentially, to vaporize phyto material 340 positioned in the corresponding region of the heating chamber 306. This may reduce the likelihood of failure as the heating unit may no longer require the heating element assemblies to be moveable.

In some embodiments, as the user inhales for a duration D_{I}, each heating element 312 can be energized sequentially for a portion of the duration D_{I} to vaporize the loose leaf phyto material 340 positioned in the corresponding region. The portion of the duration D_{I} that each heating element assembly is energized is typically the duration D_{I} divided by the number (N) of heating element assemblies 312 (for e.g., D_{I/N}). For example, if the duration D_{I} of the inhalation is 5 seconds, each heating element assembly, 312a-312j, can be energized for a duration D_{I/N} of 0.5 seconds, starting from heating element assembly 312a and ending with heating element assembly 312j. In other embodiments, each heating element assembly 312a-j may be energized for a different portion of the duration DT. For example, with a DT of 4 seconds, heating element assemblies 312a and 312b may be sequentially energized for 1 second, respectively, and then heating element assemblies 312c-312j energized sequentially for 0.25 seconds, respectively. The control circuit 314 can include a memory component (not shown) that can store user preferences for determining which heating element assembly 312 is energized at a given time.

In some embodiments, a memory component of the control circuit 314 can store data on which heating element assembly 312 has been energized. In this way, upon a subsequent inhalation, the next heating element 312 in the sequence may be energized. This may facilitate using the vaporization device 100 across different vaporization sessions without reloading the phyto material in the chamber cavity.

In some embodiments, the air cooling assembly 124 may be moveable within the inner lid space to align the air cooling assembly 124 with the heating element or elements 312 being activated. In other case, the air cooling assembly 124 may remain stationary and may be aligned with the active heating regions by the perforated floor section that directs air to the air cooling assembly 124 from the chamber cavity 310.

Referring now to FIG. 8, shown therein is another example of a heating unit that may be used with vaporization device 100. FIG. 8 illustrates an example of a heating unit that generally corresponds to a combination of the heating units shown in FIGS. 6 and 7. Elements having similar structure and/or performing similar function as those in the example vaporization device 100 in FIGS. 1-5 are numbered similarly, with the reference numerals incremented by 300. In particular, the heating unit shown in FIG. 8 includes a plurality of segments 454a-j separated by dividers 456a-i. Additionally, a separate heating element assembly 412a-j is positioned to partially surround each of the segments 454a-j, respectively.

In embodiments where the heating element assembly 412 includes a plurality of heaters (for e.g., 412a-j) positioned along the chamber length L_{C}, the control circuit 414 can control which of the heaters is energized at a given time.

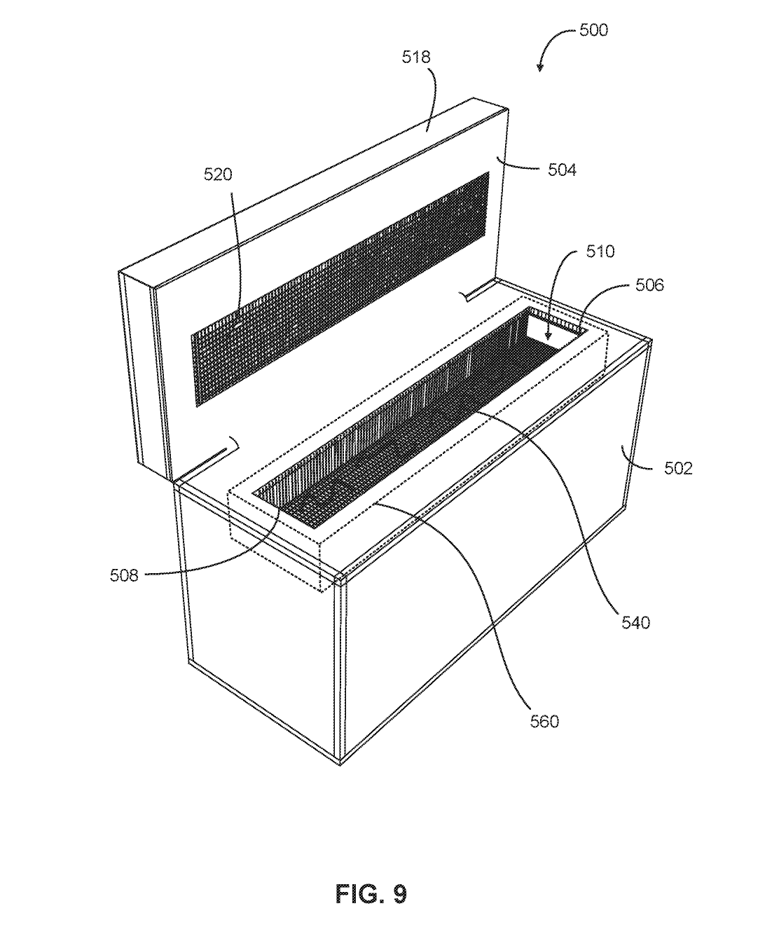

Referring now to FIG. 9 shown therein is another example of a vaporization device 500. Vaporization device 500 is generally similar to vaporization device 100 except that vaporization device 500 has been modified to incorporate a scale 560. Elements having similar structure and/or performing similar function as those in the example vaporization device 100 in FIGS. 1-5 are numbered similarly, with the reference numerals incremented by 400.

In the example shown, the body 502 of vaporization device 500 incorporates a scale 560. The scale 560 can be used to weigh phyto material 540 loaded into the chamber cavity 510 of the heating chamber 506. The scale 560 may be electrically coupled to the control circuit 514 and powered by the energy storage module 516. The scale 560 can be used to weigh the phyto material 540 as it is loaded into the chamber cavity 510.

The scale 560 may allow phyto material to be loaded into each segment (for e.g., segments 254a-j of FIG. 6) until a predetermined dose is reached. Once the predetermined dose is reached, the vaporization device 500 may provide an output signal (e.g. visual or audible) indicating that loading is complete. The scale 560 can then be tarred and another segment can be loaded to the predetermined dose.

In some cases, the scale 560 may be usable to weigh each region independently. This may facilitate loading of multiple regions simultaneously.

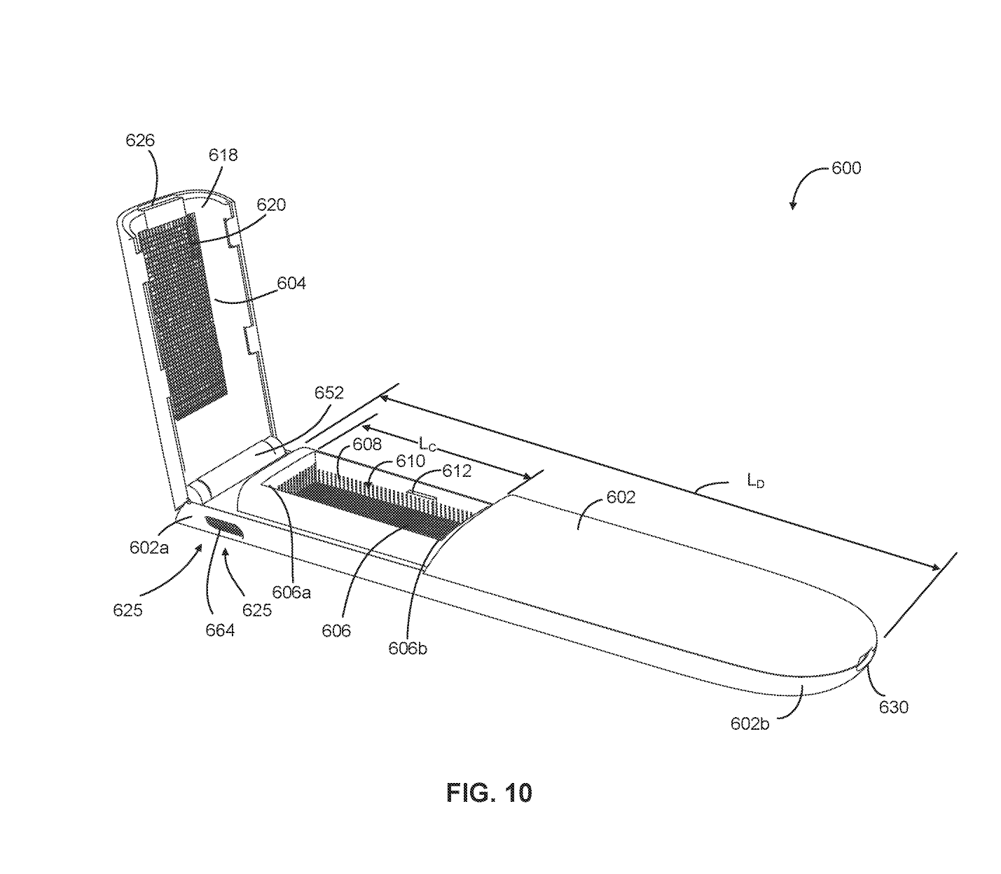

Referring now to FIGS. 10-15, shown therein is another example of a vaporization device 600. Vaporization device 600 is generally similar to vaporization device 100, although the device body 602 has been modified slightly. Elements having similar structure and/or performing similar function as those in the example vaporization device 100 in FIGS. 1-5 are numbered similarly, with the reference numerals incremented by 500.

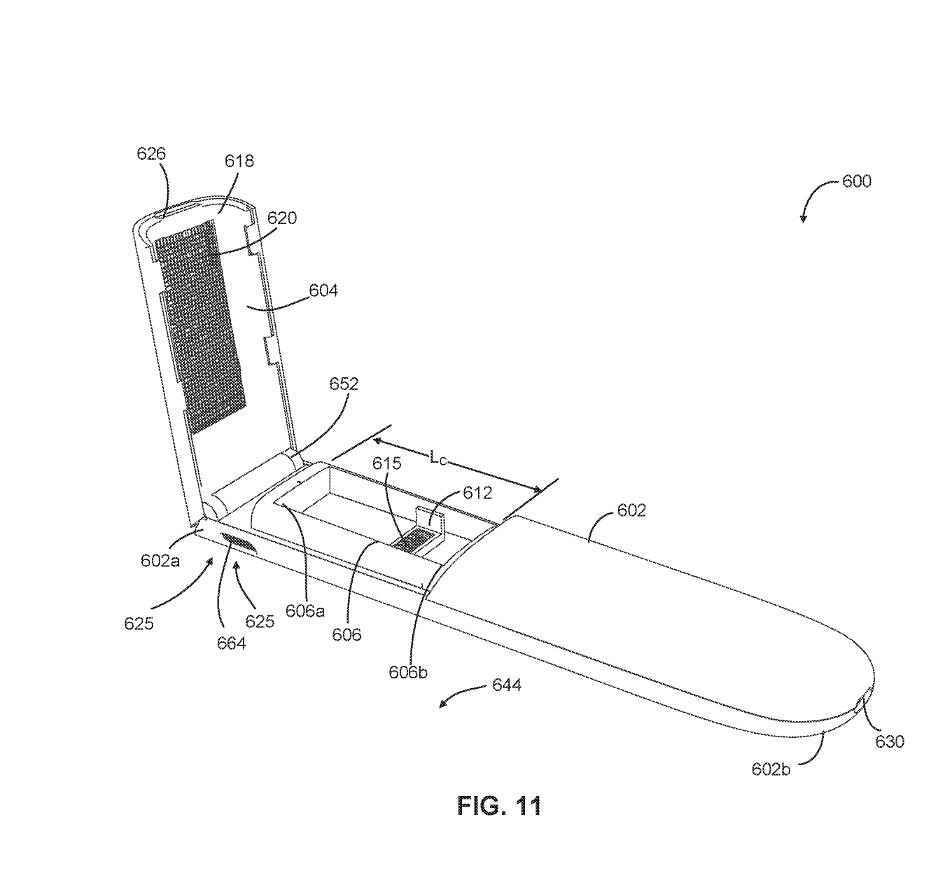

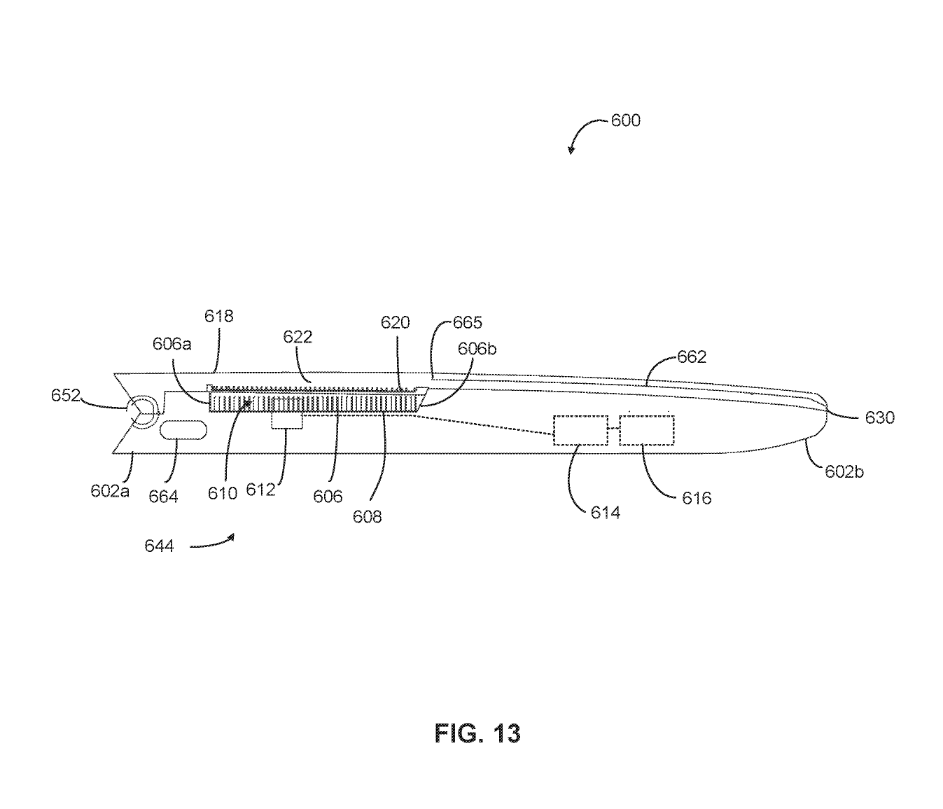

FIG. 10 shows the vaporization device 600 having an elongated device body 602 and a device lid 604 movably mounted to the elongated device body 602. The elongated device body may have a first end 602a and a second end 602b opposite the first end 602a. In the example shown, the lid 604 is movably mounted to the elongated device body 602 by a hinged connection 652 at the first end 602a. Although the hinged connection 652 is perpendicular to a length L_{D} of vaporization device 600, it will be appreciated that a parallel, or other, hinged connections are possible. The lid 604 is moveable between an open position (FIGS. 10 and 11) and a closed position (FIG. 13).

A first end section of the elongated body device 602 can include a heating unit that includes a heating chamber 606 and a heating element assembly 612. A second end section of the device body 602 may include the energy storage module and control circuit of vaporization device 600.

The heating element assembly 612 can be positioned adjacent to the heating chamber 606 (see e.g. FIG. 11). The heating chamber 606 may extend along a chamber length L_{C} from a first end 606a to a second end 606b. The heating chamber 606 can also include one or more sidewalls 608 extending from the first end 606a to the second end 606b. The sidewalls can define a chamber cavity 610 of the heating chamber 606. As shown in FIG. 10, the sidewalls 608 may be perforated sidewalls.

Referring to FIG. 11, the heating element assembly 612 can be positioned at least partially surrounding the exterior of the heating chamber 606. The heating element assembly 612 can be energized to emit heat. In the example shown, the heating element assembly 612 includes a coil heating element 615. The coil heating element 615 may be activated by directing current through the coil 615. The coil 615 may then emit heat. Heat from the heating element assembly 612 can radiate into the heating chamber 606 to heat phyto material in the chamber cavity 610 to a predetermined vaporization temperature. Phyto material vapor can then be emitted.

The lid 602 may include an outer wall 618 and a perforated floor surface 620. A lid inner space 622 (FIG. 13) may be defined between the outer wall 618 and the perforated floor surface 620. When in the open position, the inner lid space 622 may be in fluid communication with the external environment, indicated generally as 644, via an air outlet 626 defined in the lid 604.

Ambient air 625 may pass into a heating chamber 606 via one or more air inlet ports defined on the device body 602. In the illustrated example, the device body has one air input port 664 defined on the first end 602a of elongated device body 602. It will be appreciated that various other configurations of the air inlet ports 664 may be possible.

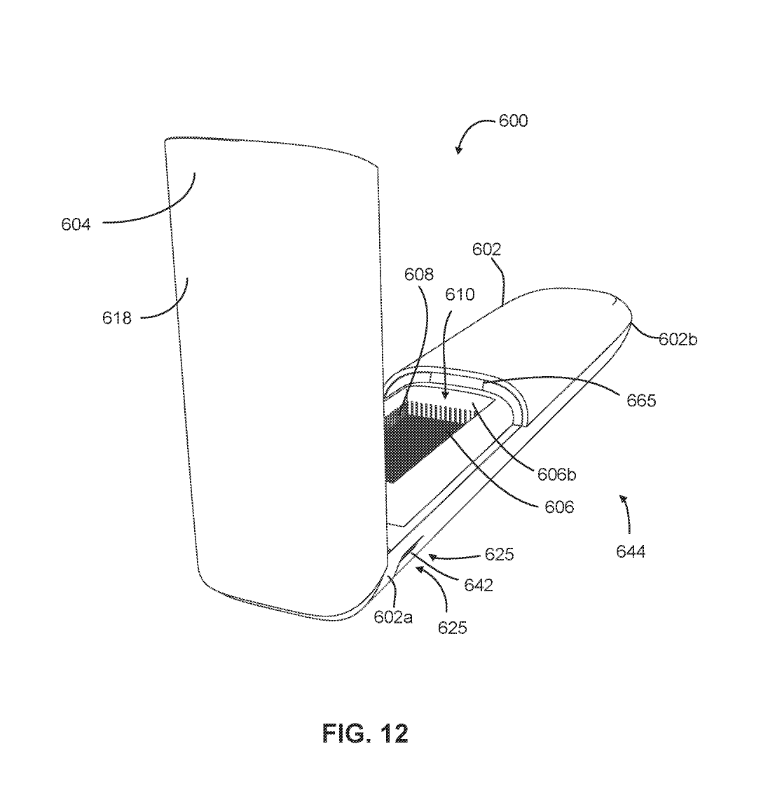

Referring to FIG. 13, the second end 602b of the elongated device body 602 may define a flow channel 662. An inhalation aperture 630 may be defined on the second end 602b of the elongated device body 602. As best seen on FIG. 12, a channel inlet 665 may also be defined on the second end 602b of the elongated device body 602. The inhalation aperture 630 and the channel inlet 665 may be in fluid communication with the flow channel 662. In the illustrated example, the channel inlet 665 and the inhalation aperture 630 are defined opposite each other on the second end section 602b of the elongated device body 602, although this need not be the case. When the lid 604 is in the closed position, at least a portion of the air outlet 626 defined in the lid and at least a portion of the channel inlet 665 defined on the elongated device body 602 align. As a result, the flow channel 662 can be fluidly connected with the lid inner space 622.

Continuing to refer to FIG. 13, the second end 602b of the elongated body device 602 may include an energy storage module 616 such as a battery electrically coupled to heating element assembly 112. The second end 602b of the elongated body device 602 may also include a control circuit 614 electrically coupled to the heating element assembly 612 and/or energy storage module 616. Control circuit 614 and energy storage module 616 may function is the same manner as control circuit 114 and energy storage module 116. In some cases, the control circuit and/or the energy storage module can be housed in the first end 602a of the elongated body device 602.

When a user inhales from the inhalation aperture 630, ambient air 625 may be drawn from the external environment 644, into the chamber cavity 610 through the one or more air inlet ports 664 and the perforated sidewalls 108. While in the chamber cavity 610, ambient air 625 may mix with vaporized phyto material and can be drawn by the inhalation into the lid inner space 622 through the perforated floor 620. The mixture may then be drawn out of the lid inner space 622 via the air outlet 626 where it may pass through the flow channel 662 before exiting at the inhalation aperture 630.

As the mixture of ambient air and vapor travels through the flow channel 662 from the channel inlet 665 to the inhalation aperture 630, the mixture may cool. This may allow a user to inhale vapor at a lower temperature.

Although not shown, the heating chamber 606 may be divided into a plurality of segments as discussed above and shown with reference to FIGS. 6 and 8.

Although not shown, the heating element assembly 612 may include a plurality of heating element assemblies positioned along the length of the heating chamber 606 as discussed above and shown with reference to FIGS. 7 and 8.

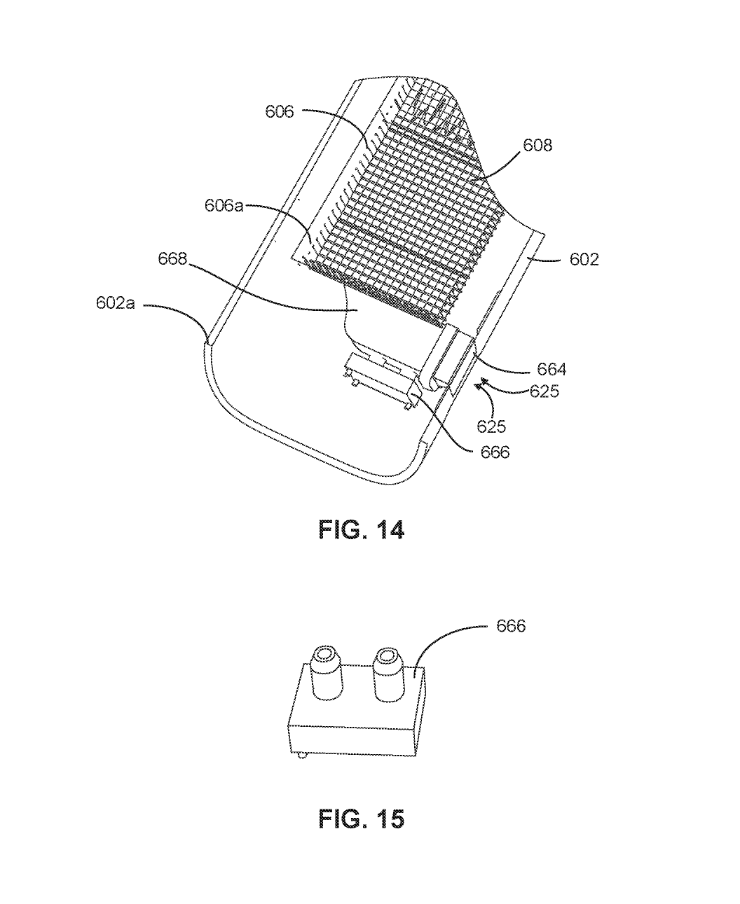

Referring to FIGS. 14 and 15, optionally, an airflow sensor 666 may be incorporated into vaporization device 600. Air flow sensor 666 may be fluidly connected to the air flow path to measure a volume of air entering the vaporization device 600 through the one of more air inlets ports 664. FIG. 14 shows a partial cutaway of the vaporization device 600 with the mass airflow sensor 666 coupled to an air intake manifold 668 that is then coupled with the heating chamber 606. That is, during an inhalation, ambient air 625 may enter through the one or more air inlet ports 664 and pass into the air intake manifold 666 before entering the heating chamber 606.

For example, a mass airflow sensor or a volumetric airflow sensor may be used to measure the airflow passing through vaporization device 600. An example mass airflow sensor similar to the one illustrated in FIG. 15 is manufactured by Sensirion, such as the SPD3x.

Alternatively, a puff sensor (not shown) may be used to determine the volume of air entering the vaporization device 600 through the one or more air inlet ports 664. An example of a puff sensor may be a microphone or a MEMS based micro capacitive type sensor. Typically, the puff sensor can be positioned with a fluid conduit aligned parallel to the flow of ambient air 625 entering the one or more air inlet ports 664. A secondary puff sensing flow path may be coupled to the inhalation aperture to determine a volume of air being drawn into the vaporization device 600. For example, the control circuit 614 may estimate the volume of airflow entering the vaporization device 100 through a stored lookup table generated by an initial calibration process (for e.g., during manufacturing).

The air flow sensor may detect a mass and/or volume of air entering the one or more air inlet ports 664 of the vaporization device 600. Optionally, the control circuit 614 may provide an airflow notification to the user which identifies for the user the mass and/or volume of air entering the vaporization device 600. Optionally, the control circuit 614 may be configured to enable and/or disable operation of the heating element assembly 612 after a predetermined mass of air and/or a predetermined volume of air has entered the vaporization device 600.

In some cases, control circuit 614 may be configured to activate one or more heating elements in response to the air flow sensor detecting an inhalation. This may allow the vaporization device 600 to reduce the draw on the energy storage module when the device 600 is not in use, by only activating the heating elements when a user is inhaling.

Referring now to FIGS. 16-20, shown therein is another example of a heating unit 700. In heating unit 700, the heating chamber 706 has a substantially cylindrical shape. A semi-annular heating element assembly 712 is positioned to partially surround the heating chamber 706. The heating element assembly 712 may be moved along the length of the heating chamber 706 in a manner similar to heating element assembly 112 described herein above.

Referring to FIG. 16, the cylindrical heating chamber 706 may have a first end 706a, a second end 706b opposite the first end 706a, and a perforated outer wall 708 extending between the first and second ends 706a and 706b. The perforated outer wall 708 may define a substantially cylindrical chamber cavity 710. In the illustrated example, the perforated outer wall 708 can be formed of a perforated wire mesh. It will be appreciated that the perforated outer walls 708 may be formed of other suitable materials.

As shown in FIG. 16, the heating chamber 706 may further include a removable cap 770 and a stopper or plug 772. The plug 772 may be inserted or connected at the second end 706b of the heating chamber 706 to seal the second end 706b from the external environment, indicated generally as 744.

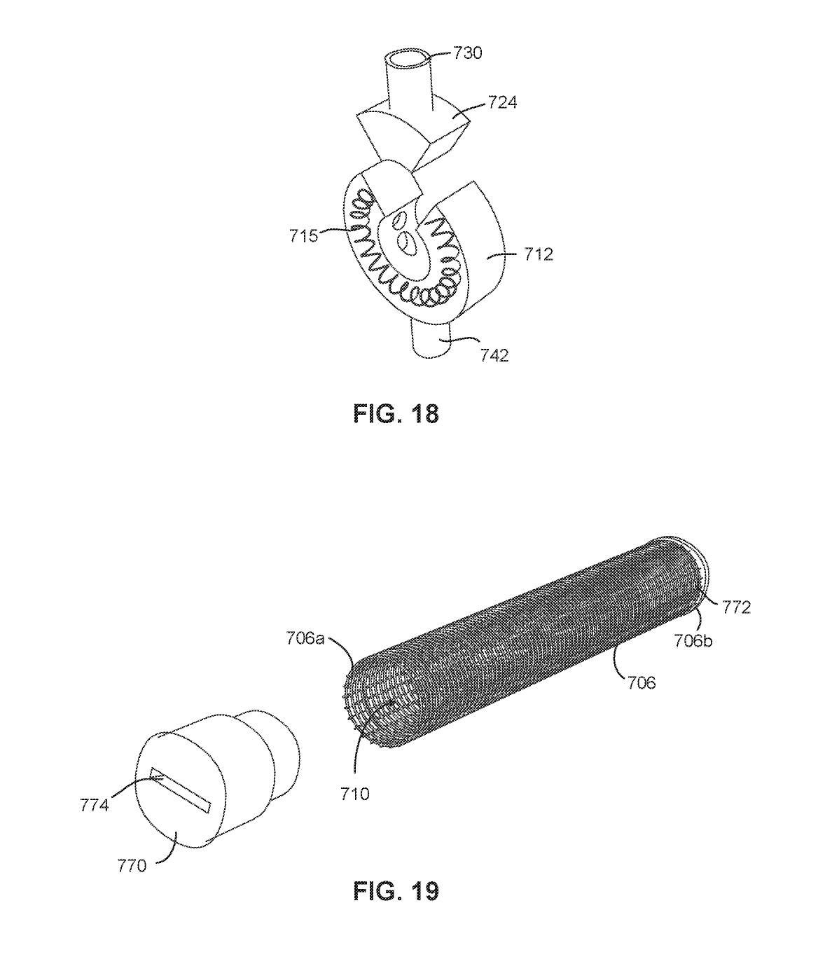

The removable cap 770 may be removably mounted or inserted at the first end 706a of the heating chamber 708. FIG. 18 shows the heating chamber 706 in a closed position with the removable cap inserted at the first end 706a of the heating chamber 708. FIG. 19 shows the heating chamber 706 in an open position with the removable cap removed from the first end 706a of the heating chamber 706. In the open position, the chamber cavity 710 may be loaded, through the first end 706a of the heating chamber 706, with phyto material for vaporization. The removable cap 770 may include an air slit 774 formed therein. In the closed position (FIG. 16), the air slit 774 can allow ambient air 725 to pass from the external environment 744 into the heating chamber 706 at the first end 706a.

An air cooling assembly 724 may be integrated with (and thermally insulated from) the heating element assembly 712. Preferably, the air cooling assembly 724 and the heating element assembly 712 form a closed annular shape with the heating chamber 706 is defined therewithin (see e.g. FIG. 16).

Referring to FIG. 16, the air cooling assembly 724 may include an outer wall 776, an inner cooling space (not shown) defined by the outer wall 776 and an inhalation aperture 730 defined on the outer wall 776 and fluidly connected to the inner cooling space. In the example illustrated, the inhalation aperture 730 can include a mouthpiece 730 that extends from the outer wall 776 of the air cooling assembly 724.

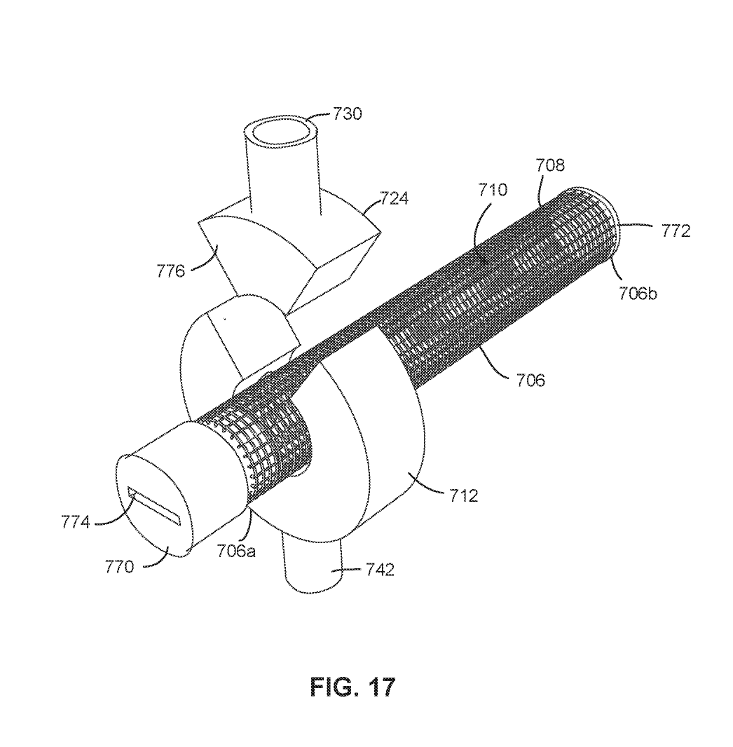

As shown in FIG. 17, the air cooling assembly may be removable from the heating unit 700. The air cooling assembly 724 may then be cleaned to remove any phyto material residue within the inner cooling space. Phyto material residue may build up over time in the inner cooling space (i.e. after repeated vaporizations) and interfere with air flow through the air cooling assembly 724. Alternatively, the air cooling assembly 724 may be replaced with a replacement air cooling assembly.

The heating element assembly 712 may also include one or more air input ports defined therein to allow ambient air to pass through the heating element assembly 712 and into the heating chamber 706. In the example illustrated, the heating element assembly 712 includes one air input port 742 that extends outwardly from heating element assembly 712. When a user inhales from inhalation aperture 730, ambient air 725 is drawn through the air input port 742 and passes into the chamber cavity 710 via the perforated outer wall 708 of the heating chamber 706. As shown, the air input port 742 is defined in the heating element assembly 712 so that it is aligned with the inhalation aperture 730 of the air cooling assembly 724. This aligned configuration may assist in directing the ambient air 725 through the chamber cavity 710 to facilitate mixing of the ambient air with vaporized phyto material.

FIG. 18 shows the heating element assembly 712 and the air cooling assembly 724 with an outer housing layer removed. As shown, the semi-annular heating element assembly 712 can include a coil heating element 715 that extends around the semi-annular heating element assembly 712. The coil heating element 715 may emit heat into the portion of the heating chamber 706 that it partially surrounds.

The outer housing layer of the air cooling assembly 724 may include thermal insulation. This can prevent heat emitted from the coil heating element 715 from passing into the inner cooling space 778. The outer insulating layer of the semi-annular heating element assembly 712 may assist in directing the heat emitted from coil heating element 715 to the portion of the heating chamber 706 that it partially surrounds.

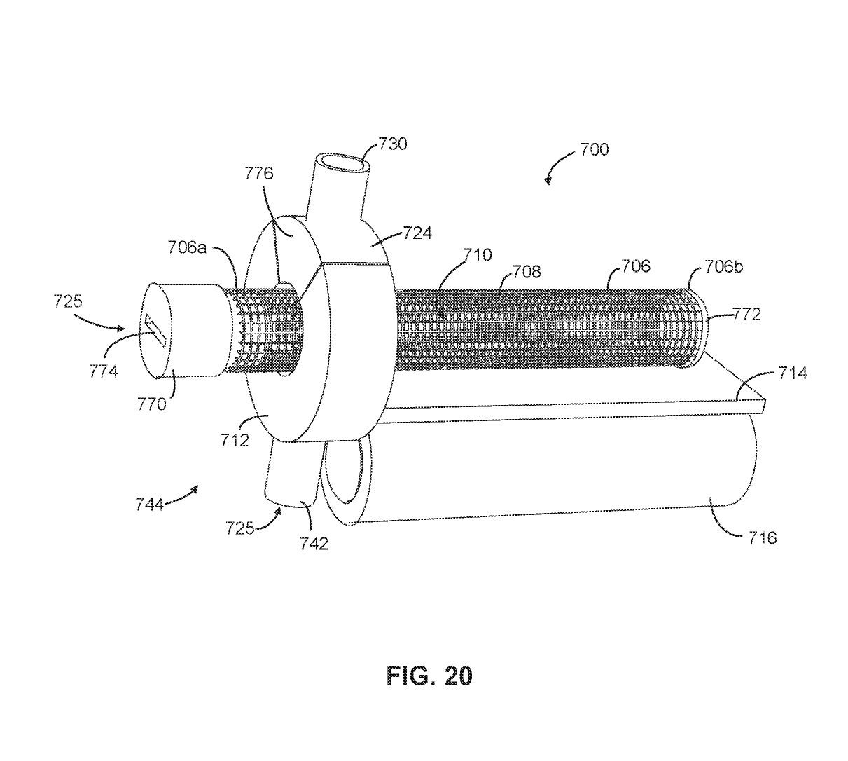

FIG. 20 shows the heating unit 700 electrically coupled to an energy storage module 716 such as a battery. Energy storage module 716 may be used to energize heating element assembly 712 to heat phyto material within the chamber cavity 710. The heating unit 700, energy storage module 716, and control circuit 716 may be enclosed within a housing (not shown).

The heating unit 700 may also include a control circuit 714 electrically coupled to the heating element assembly 712 and/or energy storage module 716. The control circuit 716 can control the operation of the heating element assembly 712. The control circuit 714 may be used to activate/deactivate the heating element assembly 712. The energy storage module 716 and the control circuit 714 may operate in a manner similar to the energy storage module 116 and the control circuit 114 described herein above.

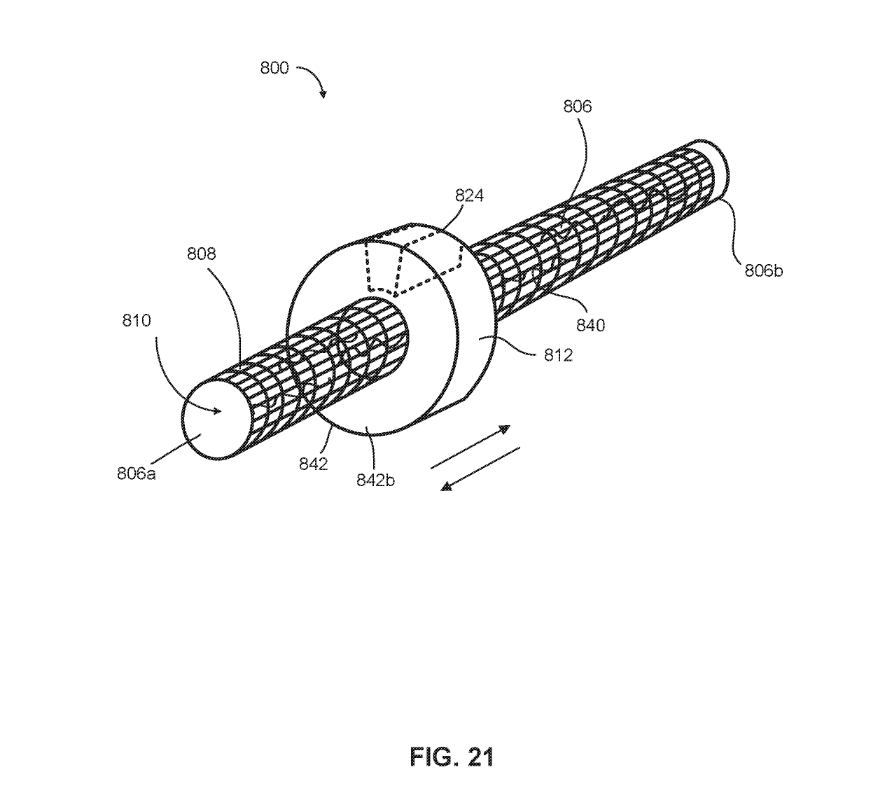

Referring to FIG. 21, shown therein is another example of a heating unit 800. The heating unit 800 shown in FIG. 21 may be generally similar to the heating unit 700 shown in FIGS. 16-20, except for slight modifications discussed herein below. Elements having similar structure and/or performing similar function as those in the example heating unit 700 in FIGS. 16-20 are numbered similarly, with the reference numerals incremented by 100.

The heating element assembly 812 may include one or more air input ports defined therein to allow ambient air to pass through the heating element assembly 812 and into the heating chamber 806. In the example illustrated, the heating element assembly 812 includes two air input port 842a and 842b. During an inhalation, ambient air 825 is drawn through the air input ports 842a and 842b and passes into the chamber cavity 810 via the perforated outer wall 808 of the heating chamber 806.

The air cooling assembly 824 may include an outer wall 876 and an inner cooling space (not shown) defined by the outer wall 876. In some embodiments, the air cooling assembly 824 may further include an inhalation aperture (not shown) defined on the outer wall 876 and fluidly connected to the inner cooling space. The air cooling assembly 824 is an example of an air cooling assembly in which the inhalation aperture is flush with the surface.

In heating unit 800, the heating chamber 806 has an open first end 806a and a closed second end 806b. Phyto material may be added into the heating chamber 806 from the open first end 806a. In some cases, the phyto material can be loaded in such a way that the phyto material is evenly distributed across the chamber of the heating chamber 806 some. In some cases, phyto material may not be evenly distributed across the length of the heating chamber 806. That is, the phyto material may loaded in different doses along the length of the heating chamber 806. In some embodiments, after loading the phyto material, the first end 806a of the heating chamber 806 may than be capped (see e.g., cap 770).

Although not shown, the heating chambers 706 and 806 of heating units 700 and 800, respectfully, may be divided into a plurality of segments as discussed above and shown with reference to FIGS. 6 and 8.

Although not shown, the heating element assemblies 712 and 812 of heating units 700 and 800, respectfully, may include a plurality of heating element assemblies positioned along the length of the heating chamber 606 as discussed above and shown with reference to FIGS. 7 and 8.

In some embodiments, inhaling at the inhalation aperture (for e.g., inhalation apertures 130, 630, 730) may include multiple inhalations. Inhalations can continue until the loose leaf phyto material (for e.g., loose leaf phyto material 140, 240, etc.) in the selected region or segment is spent (i.e. entirely vaporized).

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

1. A vaporization device for phyto material, the vaporization device comprising:
a device body comprising:
a heating chamber having a chamber wall defining a chamber cavity, the chamber wall including a perforated wall section;
a heating element assembly positioned adjacent to the perforated wall section, the heating element assembly having an external air inlet fluidly connected to an external environment;
a heating element flow path that extends from the air inlet to the chamber cavity via the perforated wall section;
a control circuit electrically coupled to the heating element assembly; and
an energy storage module electrically coupled to the control circuit; and
a lid movably mounted to the device body, the lid movable between an open position and a closed position, the lid comprising:
an outer wall;
a lid floor having a perforated floor section;
an inner lid space defined between the outer wall and the lid floor;
an air cooling assembly positioned within the inner lid space at least partially overlying the perforated floor section; and
an inhalation aperture defined in the outer wall, the inhalation aperture fluidly coupled to the air cooling assembly downstream from the air cooling assembly;
wherein,
in the open position, the chamber cavity is open to the external environment and the phyto material is loadable within the chamber cavity;
in the closed position, the lid and the heating chamber enclose the chamber cavity, and at least a portion of the perforated floor section overlies the chamber cavity, whereby the chamber cavity and the inner lid space are fluidly connected;
in the closed position, the heating element assembly is energizable to heat a defined region of the chamber cavity to a predetermined vaporization temperature; and
the air cooling assembly is alignable with the heating element assembly to define a vapor flow path from the chamber cavity through the perforated floor to the air cooling assembly and the inhalation aperture.

2. The vaporization device of claim 1, wherein:
the heating chamber has a heating chamber length;
the heating element assembly has a heating element length that is less than the heating chamber length; and
the heating element assembly is moveable along the chamber length to heat the defined heating region of the chamber cavity.

3. The vaporization device of claim 2, wherein:
the air cooling assembly has a cooling element length that is less than the heating chamber length; and
the air cooling assembly is moveable within the inner lid space, such that, in the closed position, the air cooling assembly is alignable with the defined heating region.

4. The vaporization device of claim 3, wherein, in the closed position, movement of the heating element assembly and the air cooling assembly is synchronized.

5. The vaporization device of claim 1, wherein the chamber cavity is divided into a plurality of segments along the chamber length.

6. The vaporization device of claim 5, wherein the plurality of segments are separated by at least one divider.

7. The vaporization device of claim 6, wherein a plurality of dividers are positioned within the heating chamber at regular intervals along the chamber length.

8. The vaporization device of claim 5, wherein:
each segment defines a phyto material receiving area sized to receive a predetermined volume of phyto material;
the heating element assembly is moveable along the chamber length to heat a defined segment of the chamber cavity; and
the segments are separately heatable as the heating element is moved along the chamber length.

9. The vaporization device of claim 1, wherein the heating element assembly comprises a plurality of heaters positioned along the chamber length, each heater aligned with a respective heating region.

10. The vaporization device of claim 9, wherein each heater is independently energizable to heat the respective heating region of the chamber cavity aligned with that heater.

11. The vaporization device of claim 1, wherein the chamber cavity is rectangular.

12. The vaporization device of claim 1, further comprising an airflow sensor fluidly coupled to the heating element flow path between the air inlet and the chamber cavity.

13. The vaporization device of claim 11, wherein:
the perforated wall section defines a base and two sidewalls of the rectangular chamber cavity;
the heating element assembly comprises a u-shaped heater shaped to at least partially surround the base and the two sidewalls defined by the perforated wall section.

14. The vaporization device of claim 13, wherein the u-shaped heater is positioned in the heating element flow path between the air inlet and the chamber cavity, and the u-shaped heater includes heating element outlets facing each of the base and the two sidewalls of the heating chamber.

15. The vaporization device of claim 1, wherein:
the heating chamber is cylindrical; and
the heating element assembly comprises a semi-annular heater.

16. The vaporization device of claim 1, wherein the device body further comprises a scale coupled to the chamber cavity that is arranged to weigh phyto material loaded into the chamber cavity.

17. The vaporization device of claim 1, wherein:
the vaporization device has a first end and an opposed second end;
the lid extends from the first end to the second end;
the heating chamber has a heating chamber length that extends at least partially from the first end to the second end;
the heating element assembly is moveable along the chamber length to heat the defined heating region of the chamber cavity;
the air cooling assembly is moveable within the inner lid space;
each of the heating element assembly and the air cooling assembly is moveable toward the inhalation aperture in response to a user inhaling through the inhalation aperture.

18. The vaporization device of claim 1, wherein:
the vaporization device has a first end and an opposite second end;
the lid extends from the first end to the second end;
the heating chamber has a heating chamber length that extends at least partially from the first end to the second end;
a plurality of heaters positioned along the chamber length, each heater defining a respective heating region of the chamber cavity; and
each of the heaters is individually energizable in response to a user inhaling through the inhalation aperture.

19. A method of vaporizing phyto material using a vaporization device having a heating chamber with a heating chamber length, the method comprising:
loading the phyto material into a selected vaporization region of the heating chamber;
enclosing the heating chamber with a lid;
moving a heating element assembly along the heating chamber length to align the heating element assembly with the selected vaporization region;
activating the heating element assembly; and
drawing air through the heating element assembly and into the selected vaporization region.

20. The method of claim 19, wherein moving the heating element assembly along the chamber length further comprises moving an air cooling assembly along the heating chamber to align the air cooling assembly with the selected vaporization region.

21. The vaporization device of claim 19, wherein loading the phyto material into the heating chamber comprises loading a plurality of heating chamber segments with a predetermined dose of the phyto material.

22. The method of claim 19, wherein loading the phyto material into the heating chamber further comprises loading the phyto material into a heating chamber segment until an embedded scale identifies a predetermined dose.

23. The method of claim 19, wherein the method further comprises moving the heating element assembly along the heating chamber length to a plurality of selected vaporization regions and sequentially activating the heating element assembly when the heating element assembly is aligned with each selected vaporization region.

24. A method of vaporizing phyto material using a vaporization device having a heating chamber with a heating chamber length and a plurality of heaters positioned along the heating chamber length, the method comprising:
loading the phyto material into a selected vaporization region of the heating chamber;
enclosing the heating chamber with a lid;
energizing a subset of heaters from the plurality of heaters, the subset of heaters corresponding to the selected vaporization region;
drawing air through the heating element assembly and into the selected vaporization region.

25. The method of claim 24, further comprising moving an air cooling assembly along the heating chamber to align the air cooling assembly with the selected vaporization region.

26. The method of claim 24 comprising providing a mass airflow sensor disposed between the air inlet and the chamber cavity for measuring a mass of air drawn through the heating element assembly.
